(19) United States
(12) Patent Application Publication
(10) Pub. No.: US 2014/0080729 A1
(43) Pub. Date: Mar. 20, 2014

(54) OPTICAL SENSING DEVICE FOR SENSING ANALYTES AND RELATED APPARATUS AND METHODS

(71) Applicants: Sonia Grego, Durham, NC (US); Kristin Hedgepath Gilchrist, Durham, NC (US)

(72) Inventors: Sonia Grego, Durham, NC (US); Kristin Hedgepath Gilchrist, Durham, NC (US)

(73) Assignee: Research Triangle Institute, International, Research Triangle Park, NC (US)

(21) Appl. No.: 14/006,430

(22) PCT Filed: Mar. 16, 2012
(86) PCT No.: PCT/US2012/029351

§ 371 Date: Nov. 26, 2013

Related U.S. Application Data
(60) Provisional application No. 61/466,328, filed on Mar. 22, 2011.

(51) Int. Cl. G01N 33/543

(52) U.S. Cl. CPC G01N 33/54373
USPC 506/9; 506/18; 506/16

(57) ABSTRACT

An optical sensing device and associated apparatus are configured for multiplexed detection of specific analytes in fluid samples. The device has a wavelength-tunable grating-coupler configuration in which a grating is disposed on a surface of a waveguide. Different regions of the grating may be functionalized with different receptors, and may form binding-specific sensors and reference sensors. The receptors are exposed to a fluid sample utilizing a fluidic structure mounted to the device. The device utilizes evanescent waves to sense analytes bound to the waveguide surface. The evanescent wave is sensitive to changes in refractive index at (or near) the waveguide surface. Changes in refractive index occur proportionally to the mass of the bound analyte. The apparatus utilizes a tunable light source to implement swept wavelength interrogation while the input beam is held at a fixed coupling angle relative to the waveguide and grating.

### RELATED APPLICATIONS

This application claims the benefit of U.S. Provisional Patent Application Ser. No. 61/466,328, filed Mar. 22, 2011, the content of which is incorporated by reference herein in its entirety.

### TECHNICAL FIELD

The present invention relates to optical sensing of analyte binding events based on swept wavelength interrogation of receptor-functionalized grating regions of an optical waveguide device having an input grating coupler configuration.

### BACKGROUND

Devices and techniques for sensing (detecting, measuring) analytes (e.g., drugs, biomarkers of infection, contaminants, etc.) are utilized for analyses such as medical diagnosis and detection of biochemical substances in food and the environment. Current analytical approaches are either expensive, labor-intensive and/or confined to specialized laboratories (e.g. PCR or ELISA for medical diagnostics) or limited in sensitivity, choice of targetable analytes and multiplexing ability (e.g., dipstick flow immunochromatographics tests). Evanescent wave-based sensors are being investigated as an alternative to such approaches.

An evanescent-wave based sensor generally includes a transducer in the form of an optical waveguide and a layer of biochemical-sensitive receptors immobilized on a surface of the waveguide. Such a sensor may be configured to enable label-free detection of biological, biochemical or chemical substances (analytes) that adsorb, or otherwise react with, or undergo a change in concentration over the waveguide surface. When a sample medium containing the analyte is brought into contact with the biochemical-sensitive layer on the waveguide surface, the analyte causes a change in refractive index at the biochemical-sensitive layer, which affects the evanescent portion of a guided mode propagating through the waveguide. The evanescent wave extends typically a few hundred nanometers from the waveguide surface into the sample medium and provides a large degree of discrimination between interactions occurring at the surface and in the bulk medium of the sample. The transduction mechanism (a change in intensity, angular or wavelength spectra of optical fields) provides in real time information on the amount of analyte present in the sample medium. This approach is termed label-free because it does not require time-consuming conjugation of the analyte with an optical tag (typically fluorescent or phosphorescent molecules) and derives its specificity from the biochemical-sensitive layer coating the waveguide surface. Known approaches utilizing evanescent wave sensing from patterned waveguide structures (e.g. interferometers and ring resonators) exhibit great sensitivity but they are complicated in practical implementations by requirements to couple light from the laser source into the waveguide and by elaborate microfabrication requirements.

Evanescence-wave sensing based on grating couplers integrated in planar waveguides is attractive because it enables convenient free-space light coupling into the waveguide and the relatively straightforward device structure is amenable to mass production. Grating-coupler waveguide sensors have been demonstrated in a variety of configurations in the literature (incoupling, outcoupling and resonant reflective modes), as reported, e.g., in Tiefenthaler, Advances in Biosensors 2, 261-289 (1992) and U.S. Pat. No. 7,627,201 to Tiefenthaler.

While some waveguide grating sensors have been designed for high-throughput screening (e.g., U.S. Pat. No. 7,582,486) for the biological and pharmaceutical research community, a need exists to develop compact and portable biosensors for operation in the field. Grating-coupler waveguide sensors have the capability to provide rapid, sensitive and specific biochemical sensing and have been investigated for use as portable instruments to use in the field. However, grating-coupler waveguide sensors developed thus far have several disadvantages. Grating couplers operate as a sensor under a resonant condition that occurs only for a specific angle and wavelength of the incoming light. Because the resonant condition is critically dependent on the light beam incident on the sensor, the positioning and alignment of waveguide sensors featuring input grating couplers require a tight mechanical tolerance. This tolerance requirement renders impractical the desired ability to manually place the sensor in a portable optical read-out instrument. The tolerance requirement is somewhat relaxed in a configuration that utilizes two different gratings as input and output couplers on the same sensor. In this case, however, different incoupling and outcoupling angles are needed to avoid interferences, thereby requiring different grating pitches or different waveguide film thicknesses, which makes the fabrication steps more complex and less cost-effective. Also, detection of the outcoupled light from the waveguide is generally performed in the far field, on the same side of the sensor surface as the incident light. This approach increases the size and complexity of the optical read-out structure, often requires additional optical components such as lenses on the output readout, and gives rise to the need to avoid perturbations caused by reflected, scattered and different diffraction-order light beams.

There is an ongoing need for improved biochemical sensor devices capable of performing rapid recognition of analytes, such as rapid diagnosis of infections or food contamination. There is also a need for such improved devices to be easily portable and operable at locations remote from a laboratory, such as at the point-of-care (POC) in the case of health-related diagnoses or for in-situ environmental and food safety monitoring. There is also a need for such devices to be readily configurable for sensing a wide variety of target analytes such as, for example, pathogens, toxins, antibodies, chemical contaminants, pesticides, allergens, drug residues, vitamins and hormones, among others. There is also a need for such devices to be highly sensitive and specific to the analytes of interest. There is also a need for such devices to be low-cost and disposable, and for optical read-out apparatus associated with such devices to be compact and rugged. There is also a need for such devices and apparatus to be relatively simple in terms of use and configuration.

There is also a need for such devices and apparatus to be capable of multiplexed sensing. Multiplexed sensing enables one to reference the analyte detection and compensate for instrumental drifts and sample matrix effects. Multiplexing also enables the reliable identification of disease or contamination by, for example, enabling the simultaneous detection of suitable complementary targets. For example, for the diagnosis of pathogen infections, the capability of detecting in a suitable clinical sample both the antigen as well as the host antibody response would enable reliable diagnosis over a broad window of time, because the concentration of these analytes varies with time from onset of symptoms (the antigen concentration decreases as the host response antibodies increases).

### SUMMARY

To address the foregoing problems, in whole or in part, and/or other problems that may have been observed by persons skilled in the art, the present disclosure provides methods, processes, systems, apparatus, instruments, and/or devices, as described by way of example in implementations set forth below.

According to one implementation, optical sensing device for sensing analytes in a fluid sample includes an optically transparent substrate, a waveguide composed of a higher refractive-index material than the substrate, a diffraction grating formed on the waveguide, and a plurality of sensors disposed on the diffraction grating. The waveguide includes a first surface disposed on the substrate, an opposing second surface, and an optical output edge between the first surface and the second surface. The first surface and the second surface are parallel with a waveguide plane, and the optical output edge is substantially normal to the waveguide plane. The sensors are arranged in a 1×N series, where N is an integer equal to or greater than 2. Each sensor includes a plurality of receptors immobilized on the diffraction grating. At least one of the sensors is a binding-specific sensor that includes a plurality of binding-specific receptors. The diffraction grating is configured for coupling a guided mode beam into the waveguide in response to an optical input beam incident on the sensors at a guided-mode resonance condition. The guided mode beam includes N spatially distinct components that propagate along the waveguide plane from the respective sensors to the optical output edge.

In some implementations, the substrate has a refractive index ranging from 1.4 to 1.7.

In some implementations, the waveguide has a refractive index ranging from 1.5 to 3.5. In some implementations, the waveguide has a thickness ranging from 50 nm to 1000 nm. In some implementations, the waveguide is composed of silicon oxide, silicon nitride, silicon oxynitride, or a metal oxide such as, for example, titanium dioxide, tantalum oxide, zinc oxide, hafnium oxide, or aluminum oxide.

In some implementations, the optical sensing device includes an interlayer disposed on the substrate and composed of a material of lower refractive index than the waveguide and the substrate, such that the first surface of the waveguide is disposed on the interlayer. In some implementations, the interlayer is composed of silicon dioxide or an optically transparent polymer. In some implementations, the interlayer has a refractive index ranging from 1.4 to 1.7.

According to another implementation, optical sensing apparatus for sensing analytes in a fluid sample includes an optical sensing device, a wavelength-tunable light source, and a plurality of optical detector units. The optical sensing device includes an optically transparent substrate, a waveguide composed of a higher refractive-index material than the substrate and disposed on the substrate, a diffraction grating formed on the waveguide, and a plurality of sensors disposed on the diffraction grating. The waveguide lies in a waveguide plane and includes an optical output edge. The sensors are arranged in a 1×N array, where N is an integer equal to or greater than 2. Each sensor includes a plurality of receptors immobilized on the diffraction grating. At least one of the sensors is a binding-specific sensor that includes a plurality of binding-specific receptors. The diffraction grating is configured for coupling a guided mode beam into the waveguide in response to an optical input beam incident on the sensors at a guided-mode resonance condition. The guided mode beam includes N spatially distinct guided mode components that propagate along the waveguide plane from the respective sensors to the optical output edge. The wavelength-tunable light source is configured for emitting the optical input beam at a wavelength that varies over a wavelength range at a controllable wavelength-varying rate. The wavelength-tunable light source is positioned relative to the optical sensing device wherein the optical input beam propagates to the sensors at a fixed coupling angle. The optical detector units are positioned for receiving respective N output beam components outcoupled from the optical output edge. The N output beam components correspond to the N guided mode components.

According to another implementation, the wavelength-tunable light source is positioned such that the optical input beam passes through the substrate and the waveguide before irradiating the sensors.

According to another implementation, a method is provided for sensing analytes in a fluid sample. The fluid sample is brought into contact with a plurality of sensors arranged in a 1×N array on a diffraction grating of a waveguide, where N is an integer equal to or greater than 2. Each sensor includes a plurality of receptors immobilized on the diffraction grating, wherein at least one of the sensors is a binding-specific sensor. An optical input beam is directed to the sensors at a fixed coupling angle. While directing the optical input beam, the optical input beam is scanned over a range of wavelengths. At least one of the wavelengths satisfies a guided-mode resonance condition such that the diffraction grating couples a guided mode beam into the waveguide. The guided mode beam includes N spatially distinct guided mode components that propagate along the waveguide plane from the respective sensors to an optical output edge of the waveguide. N output beam components, which correspond to the N guided mode components, are outcoupled from the optical output edge and received at respective optical detector units to produce N signals. The N signals are proportional to respective intensities of the N output beam components at the scanned wavelengths. Based on the received signals, a determination is made as to whether a wavelength spectral shift in the guided mode beam has occurred. The wavelength spectral shift is indicative of a binding event occurring at the binding-specific sensor.

In some implementations, the coupling angle of the optical input beam ranges from −20° to +20° relative to an axis normal to the waveguide plane.

According to another implementation, a method is provided for detecting an infection caused by a pathogen to an organism. A physiological sample derived from the organism is brought into contact with a first sensor and a second sensor disposed on a diffraction grating of a waveguide. The first sensor includes a plurality of first receptors immobilized on the diffraction grating, and the second sensor includes a plurality of second receptors immobilized on the diffraction grating. The first receptors are configured for binding specifically to a first binding partner. The first binding partner may be the pathogen or a biomarker indicative of the presence of the pathogen. The second receptors are configured for binding specifically to a second binding partner indicative of an immunological response to the pathogen. The first sensor and the second sensor are irradiated with an optical input beam directed at a fixed coupling angle relative to the waveguide. While irradiating, the optical input beam is scanning over a range of wavelengths. A first output beam component and a second output beam component are outcoupled from an edge of the waveguide. The first output beam is generated in response to irradiation of the first sensor, and the second output beam is generated in response to irradiation of the second sensor. Intensities of the first output beam component are measured as a function of the scanned wavelengths, and intensities of the second output beam component are measured as a function of the scanned wavelengths. Based on the intensities measured, a determination is made as to whether the first receptors have captured the first binding partner and whether the second receptors have captured the second binding partner.

According to another implementation, the first binding partner is the biomarker indicative of the presence of the pathogen. The biomarker may be, for example, a nucleic acid of the pathogen, a coating protein, or a gene product of the pathogen such as structural or non-structural proteins.

According to another implementation, the second binding partner indicative of an immunological response to the pathogen is an antibody against the pathogen such as immunoglobulin M (IgM) or immunoglobulin G (IgG), a cell surface marker, a white blood cell marker, a chemokine, a cytokine, or a macrophage activation marker.

In some implementations, the first sensor and the second sensor are arranged in a one-dimensional array, i.e., a single line of sensors perpendicular to the propagation direction of the guided mode. In some implementations, the intensities of the first output beam and the second output beam are measured by respective optical detector units arranged in optical communication with the first sensor and the second sensor, respectively. The optical detector units may be positioned near the edge at which the first optical output beam component and the second optical output beam component are outcoupled and optically aligned with the respective sensors. Alternatively, optical fibers may be positioned near the edge in optical alignment with the respective sensors and utilized to guide the optical signals to the respective sensors. In some implementations, the edge at which the first optical output beam component and the second optical output beam component are outcoupled for collection by the optical detector units is parallel with the first sensor and the second sensor.

According to other implementations, the present disclosure provides various kits for carrying out the optical sensing techniques described herein. In some implementations, a kit may include one or more optical sensing devices as described herein. In other implementations, a kit may also include an optical sensing apparatus as described herein configured for use with the optical sensing devices.

Other devices, apparatus, systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The invention can be better understood by referring to the following figures. The components in the figures are not necessarily to scale, emphasis instead being placed upon illustrating the principles of the invention. In the figures, like reference numerals designate corresponding parts throughout the different views.

FIG. 1 is a cross-sectional side view of an example of an optical sensing device according to certain implementations of the present disclosure.

FIG. 2 is a perspective view of the optical sensing device illustrated in FIG. 1, and a schematic view of a portion of an optical sensing apparatus (or system) that may include the optical sensing device.

FIG. 3 is a schematic view of an example of an optical sensing apparatus (or system) that may include the optical sensing device and associated components illustrated in FIG. 2.

FIG. 4 is an example of measurement data that may be presented by the optical sensing apparatus illustrated in FIG. 3.

FIG. 5 is a schematic top plan view of an example of a grating with N sensors that may be provided by the optical sensing device illustrated in FIG. 1.

FIG. 6 is an exploded view of an example of an optical sensing device that includes a multi-channel fluidic structure.

FIG. 7 is another exploded view of the optical sensing device illustrated in FIG. 6, but with a spacer of the fluidic structure disposed on a waveguide of the optical sensing device.

FIG. 8 is a schematic view of an example of a fluid system in which an optical sensing device and fluidic structure such as illustrated in FIGS. 6 and 7 may operate.

FIG. 9 is a schematic view of a waveguide functionalized with receptors.

### DETAILED DESCRIPTION

As used herein, the term “analyte” refers to any molecule of interest capable of being detected by an optical sensing device in accordance with the mechanisms described below. Examples of analytes include, but are not limited to, proteins, carbohydrates or other biopolymers, pathogens such as viruses, bacteria, prions or fungi, antigens, haptens, antibodies (e.g., immunoglobulins), animal or anti-human antibodies (e.g., antiglobulins), cells, toxins, drugs, steroids, vitamins, peptides, hormones, allergens, pesticides, various non-biological chemicals, and fragments, particles or partial structures of any of the foregoing, and binding partners of any of the foregoing.

As used herein, the term “fluid sample” or “liquid sample” refers to any flowable substance capable of being assayed to determine whether the sample contains one or more analytes of interest, or which is known or suspected of containing such analytes. The “fluid sample” or “liquid sample” may, for example, be a bodily (human or animal) fluid (e.g., blood, serum, plasma, other fluids), a solution containing a biological tissue or cell, a solution derived from the environment (e.g., surface water, or a solution containing plant or soil components), a solution derived from food, or a solution derived from a chemical or pharmaceutical process (e.g., reaction, synthesis, dissolution, etc.).

As used herein, the term “binding partner” refers to any molecule capable of binding to another molecule, i.e., to another binding partner. Examples of molecules that are binding partners to each other include, but are not limited to, antibody-antigen, antibody-hapten, hormone-hormone receptor, lectin-carbohydrate, enzyme-enzyme inhibitor (or enzyme cofactor), biotin-avidin (or streptavidin), ligand-ligand receptor, protein-immunoglobulin, and nucleic acid-complementary nucleic acid (e.g., complementary oligonucleotides, DNA or RNA). Depending on the type of assay being implemented, a binding partner may be an analyte to be detected, or may be an intermediate binding partner utilized in various ways in the course of detecting the analyte.

As used herein, the term “receptor” refers to any binding partner that is capable of being surface-immobilized by a suitable funtionalization technique. Examples of receptors include, but are not limited to, binding partners of analytes such as those mentioned above. A receptor may be a “binding-specific” receptor (a “binding partner-specific” receptor, or “recognition-specific” receptor) or may be a “reference” receptor.

A “binding-specific” receptor is one that has a high affinity for and readily binds to a specific type of binding partner, and which under normal assaying conditions does not bind to any other type of molecule. As an example, a binding-specific receptor may be an antibody that will only bind to a specific type of antigen, antigen analog or hapten. Depending on the assay format implemented, a binding-specific receptor may be an analyte-specific receptor, i.e., may act as a direct binding partner for the analyte to be detected in a fluid sample or for a conjugate of the analyte or a complex containing the analyte. Alternatively, a binding-specific receptor may be a binding partner for another non-analyte binding partner, and that other non-analyte binding partner may in turn be a specific binding partner for the analyte to be detected.

Depending on the implementation, a “reference” receptor may be utilized in conjunction with a binding-specific receptor. A “reference” receptor is any receptor composed or configured to produce a reference signal, which may be utilized as a control to provide a reference or baseline optical measurement signal, as described below. As an example, a reference receptor may be a “non-specific” receptor, i.e., one capable of binding to a variety of different types of molecules that may be contained in the fluid sample being assayed. A reference receptor is typically not capable of binding to the same type of binding partner as the binding-specific receptor. The composition or configuration of a reference receptor may depend on the type of assay being implemented, the type of analytes to be detected, and the type of binding-specific receptors being utilized.

For convenience, terms such as “sensor” and “sensing” as used herein generally encompass terms such as biosensor, chemical sensor, biochemical sensor, and the like. In the context of the present disclosure, such terms are generally associated with a device or system configured for sensing or detecting analytes of a biological and/or chemical nature.

In the context of the present disclosure, the term “sensor” encompasses “binding-specific sensors” and “reference sensors.” A binding-specific sensor is a sensor that includes binding-specific receptors configured to capture a specific type of molecule, as noted above. A reference sensor is a sensor that includes reference receptors and may be utilized as a control or reference, as noted above.

The present disclosure describes an optical sensing device and associated apparatus (or system) configured for multiplexed detection of specific analytes in fluid samples. The optical sensing device has a wavelength-tunable grating-coupler configuration in which a diffraction grating is integrally formed on an optical waveguide. The diffraction grating is rendered (bio)chemo-sensitive by depositing a (bio)chemo-sensitive layer on its surface. This is achieved by functionalizing one or more regions of the diffraction grating with binding-specific receptors to form binding-specific sensors, or with both binding-specific receptors and reference receptors to form respective binding-specific sensors and reference sensors. The binding-specific sensors, or both binding-specific sensors and reference sensors, are exposed to a fluid sample utilizing a fluidic structure mounted to the optical sensing device. The optical sensing device utilizes evanescent waves to sense analytes (or binding partners of analytes) bound to the sensors. The evanescent wave is the fraction of propagating light that extends out from the waveguide core film into the fluid sample. The evanescent wave is sensitive to changes in refractive index at (at or near) the waveguide surface. Changes in refractive index occur proportionally to the mass of the bound analyte. This enables label-free (bio)chemical detection, as the presence of the target analyte is determined without the requirement of attaching fluorophores or chemiluminescent probes to the analyte.

In typical implementations, the optical sensing device operates in conjunction with an optical sensing apparatus that includes a light source, an optical detector, signal-processing electronics, and a device for outputting data which may, for example, include a graphical user interface (GUI). The response (analyte recognition) of the optical sensing device may be monitored on a display screen of the apparatus. The apparatus may be configured such that the response appears as a single value changing as a function of time, whereby the apparatus may be user-friendly and require a relatively low level of skill to operate. The apparatus operates as a reader of the optical sensing device. Different optical detector units (e.g. photodiodes, charge-coupled devices, etc.) respectively interrogate the different functionalized regions (sensors) of the optical sensing device. In advantageous implementations, the apparatus utilizes the wavelength spectral shift of a largely tunable laser as the transduction mechanism, as described further below. The optical sensing device and other components of the apparatus may be small and amenable to large-scale manufacture and the apparatus may be packaged compactly, and thus the apparatus may be implemented as a portable, cost-effective instrument for point-of-care diagnostics. The optical sensing device may be disposable, and the apparatus may be utilized in conjunction with different optical sensing devices configured for detecting different types of analytes and carrying out different types of assay formats.

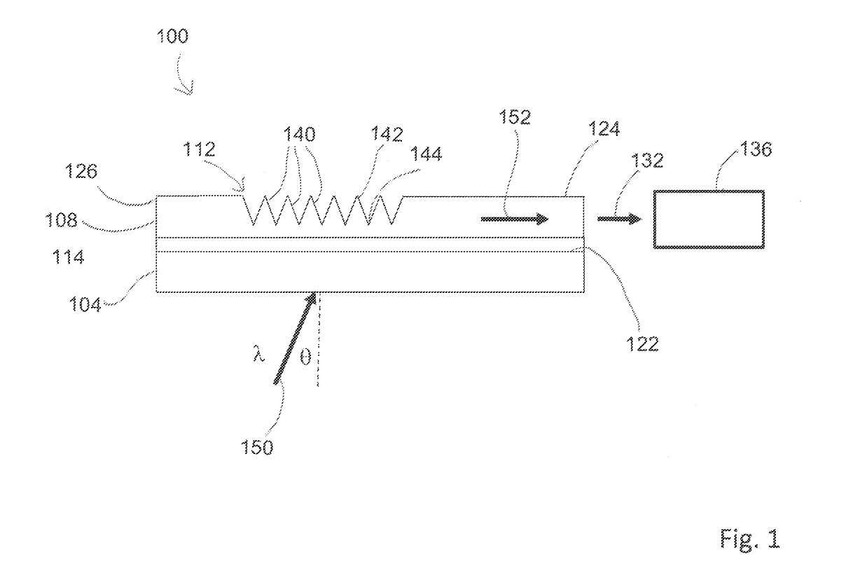

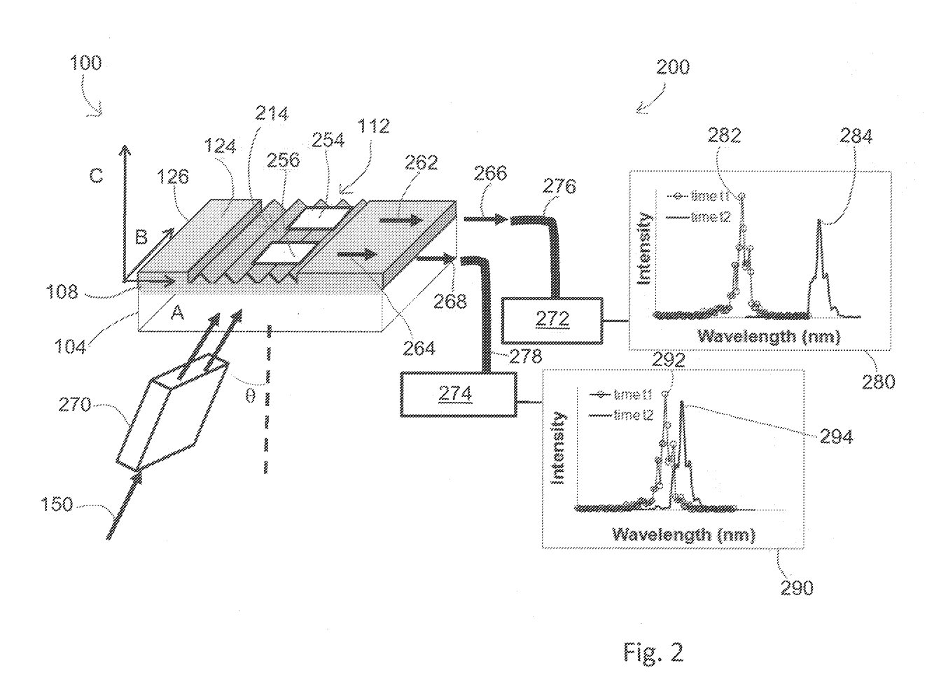

FIG. 1 is a cross-sectional side view of an example of an optical sensing device 100. FIG. 2 is a perspective view of the optical sensing device 100, and a schematic view of a portion of an optical sensing apparatus (or system) 200 that may include the optical sensing device 100. For reference purposes, FIG. 2 depicts a system of three mutually orthogonal axes A, B and C. The A and B axes lie in plane that will be referred to as the waveguide plane. The C axis will be referred to as the normal axis, which is orthogonal to the waveguide plane. The optical sensing device 100 generally includes a substrate 104, an optical waveguide 108 disposed on the substrate 104 and including an optical diffraction grating 112, and a plurality of sensors 214 disposed on the diffraction grating 112. Typically, the optical diffraction grating 112 is integrally formed on the side of the optical waveguide 108 opposite to the substrate 104, or alternatively on both sides of the optical waveguide 108. The plurality of sensors 214 are disposed on the diffraction grating 112 located on the side of the optical waveguide 108 opposite to the substrate 104.

The substrate 104 may generally be composed of any optically transparent, low refractive-index material on which the waveguide 108 may be fabricated by a typical microfabrication process. In the present context, “optically transparent” means able to efficiently pass (with minimal optical transmission loss) an optical (electromagnetic) beam of a desired wavelength λ (e.g., 1550 nm) through a given material. In the present context, a refractive index (or index of refraction) is “low” if its value is lower than the refractive index of the waveguide 108. The refractive index of the substrate 104 may range, for example, from 1.4 to 1.7. Examples of compositions suitable for the substrate 104 include, but are not limited to, silicon, glass, quartz, and certain plastics (e.g., polycarbonate, poly (methyl methacrylate) or PMMA).

In some implementations, an interlayer (an intermediate layer, or buffer layer) 114 of a low refractive-index material may be interposed between the substrate 104 and the waveguide 108. The interlayer 114 may be provided, for example, in implementations where the index of refraction of the material of the substrate 104 is not sufficiently low relative to the waveguide 108 for the wavelength λ contemplated for operation. For example, the interlayer 114 may be useful when the substrate 104 is silicon and the operating wavelength λ is 1550 nm. The refractive index of the interlayer 114 may range from, for example, 1.4 to 1.7. Examples of low refractive-index compositions suitable for the interlayer 114 include, but are not limited to, oxides such as silicon dioxide (SiO₂), and certain optically transparent polymer films. Depending on the compositions of the substrate 104 and the waveguide 108, the interlayer 114 may also be useful for facilitating deposition of the waveguide 108 on the substrate 104, e.g., to provide strain relief, prevent cracking, reduce the surface roughness of the substrate 104, reduce mismatches in the respective coefficients of thermal expansion and/or lattice constants between the substrate 104 and the waveguide 108, etc.

In typical implementations the substrate 104 and the interlayer 114 (if provided) are planar, i.e., each has a dominant area (length×width) parallel with the waveguide plane and a thickness (along the normal axis) smaller than either the length or the width. The thicknesses of the substrate 104 and the interlayer 114 may depend in part on their compositions and the desired wavelength λ that is to be efficiently passed therethrough. As non-limiting examples, the thickness of the substrate 104 may range from 0.3 to 2 mm and the thickness of the interlayer 114 may range from a few (e.g., 1-3) micrometers to a few millimeters. In certain implementations, it is advantageous for the thickness of the interlayer 114 to be at least three times the wavelength λ of the optical beam utilized. In typical implementations the substrate 104 and the interlayer 114 have rectilinear shapes.

The waveguide 108 may generally be composed of any optically transparent, high refractive-index material that may be deposited on the substrate 104 (or interlayer 114) by a typical microfabrication process. The refractive index of the waveguide 108 may range, for example, from 1.5 to 3.5. In many applications, it is preferable for the refractive index to be 1.8 or higher for enhanced sensitivity. In typical implementations the waveguide 108 is a dielectric slab. Examples of compositions suitable for the waveguide 108 include, but are not limited to, silicon dioxide (SiO₂), silicon nitride (e.g, Si₃N₄), silicon oxynitride (SiOₓN_{y}), titanium dioxide (TiO₂), tantalum oxide (Ta₂O₅), zinc oxide (ZnO), hafnium oxide (HfO₂), and aluminum oxide (Al₂O₃), other suitable optically-transparent, high refractive-index metal oxides, and combinations of two or more of the foregoing. In one example, the waveguide 108 is fabricated by depositing silicon oxynitride on a glass or silicon substrate 104 by plasma-enhanced chemical vapor deposition (PECVD) at a deposition temperature of 350° C. and low radio frequency (RF) (e.g., 100 kHz). This deposition approach enables the refractive index of the waveguide 108 to be tuned by appropriately controlling the compositions and flow rates of the precursor gases. For example, deposition of silicon oxynitride at 100 kHz may result in a waveguide 108 that exhibits low optical transmission loss, for example 0.9 dB/cm for a film having n=1.6 and 3 dB/cm for a film having n=1.65. For detection of guided mode light outcoupled at the edge of the waveguide 108, the optical loss of the waveguide should be small so that a high signal-to-noise ratio (S/N) can be achieved.

In typical implementations the waveguide 108 is planar and has the same dimensions (length and width) as the substrate 104. As a non-limiting example, the length of the waveguide 108 (along the A axis) may range from about 5 to 50 mm. The thickness of the waveguide 108 may be selected such that the waveguide 108 is single-mode. This configuration enables the propagation of the fundamental TE (transverse electric) and TM (transverse magnetic) modes, which have orthogonal polarizations. The evanescent wave sensing mechanism is most sensitive when these modes are utilized. The thickness of the waveguide 108 is typically such that the waveguide 108 may be characterized as a thin film. As a non-limiting example, the thickness may range from 50 nm to 1000 nm. In typical implementations the waveguide 108 has a rectilinear shape. The outer surfaces of the waveguide 108 include a planar first surface 122 disposed on the substrate 104 (or the interlayer 114), an opposing planar second surface 124, and a peripheral surface 126. The first surface 122 and the second surface 124 may be parallel with the waveguide plane, and the peripheral surface 126 may be substantially normal to the waveguide plane. In the present context, the term “substantially normal” means that the peripheral surface 126 (or a section thereof) may be normal to the waveguide plane (i.e., parallel with the normal axis) or may deviate from precise normality by a few degrees. A section of the peripheral surface 126 (typically defining one distinct side of the waveguide 108) is referred to herein as an optical output edge 128. The optical output edge 128 is any section of the peripheral surface 126 utilized for outputting an optical output beam 132 (i.e., optical measurement beam or signal) to an optical detector 136 as described below.

The waveguide 108 may be considered as serving as a high refractive-index optical core of the optical sensing device 100. The substrate 104 may serve as a low refractive-index lower cladding for the waveguide 108, and during operation a fluid sample (or “cover medium”) located on the waveguide 108 serves as a low refractive-index upper cladding. Alternatively the interlayer 114, when provided between the substrate 104 and the waveguide 108 as described above, serves as the lower cladding.

The diffraction grating 112 is a region on the second surface 124 of the waveguide 108 containing a periodic structure. The grating 112 is a distinct operative feature of the optical sensing device 100, and in typical implementations is integrally formed on (e.g., is a surface feature of) the waveguide 108. The grating 112 may be formed on the waveguide 108 by, for example, ultraviolet (UV) lithography, imprint lithography, holographic lithography, or embossing. In particular, thermal nanoimprint lithography (NIL) followed by dry etching has been found advantageous for its high resolution, cost-effectiveness, and high-throughput scalability. The grating 112 may alternatively be formed on both sides of the waveguide core film using other approaches. For example, the top surface of the substrate 104 may be patterned with the periodic structure, and then the waveguide core film conformally deposited on the substrate 104, whereby the periodic structure of the grating 112 is formed on both the first surface 122 and the second surface 124 of the waveguide 108.

The grating 112 may be a one-dimensional (or unidiffractive) grating as in the present example, or alternatively may be a bidiffractive or more generally a multi-diffractive grating. In the illustrated example in which the grating 112 is unidiffractive, the periodic structure of the grating 112 is in the form of a series of parallel linear grooves 140. The grooves 140 may be defined as an alternating series of parallel linear maxima 142 and minima 144. In the implementation specifically illustrated the grooves 140 have a triangular-toothed profile, although other profiles may be suitable (e.g., saw-toothed, square-toothed, trapezoidal-toothed, sinusoidal or rounded corrugations, etc.). The grooves 140 may be parallel with the shorter side of the waveguide 108 (the width along the B axis) as in the illustrated implementation, or alternatively may be parallel with the longer side of the waveguide 108 (the length along the A axis), with optical detectors located appropriately for collecting outcoupled from the designated optical output edge 128. In the illustrated unidiffractive example, the grooves 140 are parallel with the optical output edge 128 of the waveguide 108. The grooves 140 may span the entire width or length of the second surface 124 of the waveguide 108 as illustrated, or may occupy a smaller area on the second surface 124. The area of the grating 112 may range, for example, from 1 mm² to 400 mm². As another example, the size of a given side (length or width) of the grating 112 may range from 1 mm to 20 mm. As another example, for a square grating 112 the area may range from 1 mm×1 mm to 20 mm×20 mm. The depth of the grooves 140 (distance between the maxima 142 and minima 144 along the normal axis) may range, for example, from 10 to 300 nm. The pitch (or periodicity) A of the grooves 140 may range, for example, from 250 nm to 2000 nm, with about 1000 nm being preferred in many implementations. Alternatively, a one-dimensional array of physically separate gratings 112 may be provided for defining individual sensors, or a light absorbing or masking structure may be placed on the grating 112 or along the guided mode propagation path.

In cases where the grating 112 is multi-diffractive, various configurations for the periodic structure may be implemented in various patterns or arrangements, and at a variety of angles relative to the edges of the waveguide 108 such that the guided modes are outcoupled at more than one edge of the waveguide 108. Hence, any edge of the waveguide 108 may serve as the optical output edge 128 and a suitable optical detector 136 may be located at that edge. As an example, a multi-diffractive grating may be formed by a pattern of pillars (or posts, mesas, etc.) formed on the waveguide 108.

In either unidiffractive or multi-diffraction configurations, the distance between the grating 112 and the edge of the waveguide 108 serving as the optical output edge 128 where the guided mode output beam 132 is outcoupled and collected, may range from 1 to 40 mm, with 5 to 10 mm being preferable in many implementations. Generally, this distance is the smallest distance along the waveguide plane between the structural features of the grating 112, whether grooves 140, pillars or otherwise, and the optical output edge 128.

More generally, the grating 112 is configured to operate as an optical input coupler. When an optical input beam (excitation beam) 150 incident on the grating 112 propagates at a specific resonance angle (or coupling angle) θ relative to the normal axis and at a specific wavelength λ satisfying the guided-mode resonance condition (which also depends on the structure of the waveguide 108 and the grating 112), the grating 112 efficiently couples the optical input beam 150 into the material of the waveguide 108. A resulting guided mode beam 152, guided by total internal reflection, propagates from the grating 112, through the waveguide 108 and to the optical output edge 128 along the waveguide plane. A corresponding optical output beam 132 is then outcoupled from the optical output edge 128. Upon being emitted from the optical output edge 128, the optical output beam 132 may be collected by a suitable optical detector (e.g., a photodetector) 136 for measurement of its intensity as described below. A peak in the intensity is indicative of resonant coupling at the wavelength λ corresponding to that peak. Thus, for example, when no binding events have occurred at the receptors of the sensors 214, the measured wavelength peak may indicate that the guided-mode resonance condition is fulfilled at a wavelength λ₁. Subsequently, when binding events have occurred at the receptors of the sensors 214, the measured wavelength peak may indicate that the guided-mode resonance condition is no longer fulfilled at the wavelength λ₁ and is now fulfilled at a different wavelength λ₂. Observing the shift in the wavelength peak (and accordingly the shift in the resonance condition) provides an indication of the occurrence of the binding events.

While most of the in-coupled light propagates as the guided mode beam 152, a portion of the light—the evanescent wave field (not shown)—penetrates a small distance below the waveguide 108 (into the substrate 104, or the interlayer 114 if present) and above the waveguide 108. The evanescent wave field penetrates far enough beyond the second surface 124 of the waveguide 108 to irradiate the layer of sensors 214 where binding events occur. The intensity of the evanescent wave field drops exponentially with increasing distance above the second surface 124, and become negligible at a distance of less than half of the wavelength λ of the in-coupled light. The decay length L of the evanescent wave field is typically 100 to 200 nm.

In the present implementation, the sensors 214 are arranged on the grating 112 in a 1×N array (i.e., a one-dimensional line of N sensors 214). In some implementations, the 1×N array is parallel with the optical output edge 128. In the present implementation, N is an integer equal to or greater than 2, of which at least one binding-specific sensor is provided. Depending on the type of assay to be performed, all N sensors 214 may be binding-specific sensors, or at least one of the sensors 214 may be a reference sensor. In FIG. 2 two individual sensors 254, 256 are illustrated as an example, one binding-specific sensor 254 and one reference sensor 256. The binding-specific sensor 254 may be a layer of binding-specific receptors immobilized on a first region of the grating 112, and the reference sensor 256 may be a layer of reference receptors immobilized on a second region of the grating 112. Thus, each sensor 254, 256 is a region of the grating 112 that has been functionalized with a particular type of receptor. Differently functionalized sensors 254, 256 nonetheless have identical grating properties. The receptors may be immobilized either directly on the grating 112 as a two-dimensional layer, or as a three-dimensional matrix that extends for a distance not greater than the penetration depth of the evanescent wave. The density of the receptors affects the sensitivity of the optical sensing device 100, and a greater density may be achieved when the receptors are immobilized as a three-dimensional matrix. The sensor areas cover at least a portion of the grating 112. The sensor areas may extend over one or more boundaries of the grating 112 and cover a portion of the second surface 124 of the waveguide 108. The sensors 254, 256 may generally have rectilinear shapes as in the illustrated example, or may have any other suitable shapes (e.g., dots, irregular shapes, etc.).

Depending on the type of receptor utilized, various surface functionalization techniques may be utilized to deposit the receptors on the grating 112 as appreciated by persons skilled in the art, including for example physisorption, electrostatic interaction, covalent coupling, or biotin-avidin coupling. Depending on the type of receptor utilized, the receptors may be bound or attached with or without the inclusion of one or more binding agents or linker molecules (or cross-linkers). For example, the grating 112 may need to be silanized to enable the subsequent immobilization of certain receptors on the grating 112. In one specific example, the grating 112 may be silanized with aminosilane and then exposed to a solution of maleimidopropionic acid N-hydroxysuccchinimide (NHS)-biotin, with the biotin serving as an analyte-specific receptor for the proteins streptavidin or avidin. In another specific example, the grating 112 may be silanized with glycidopropyltrimethoxysilane (GPTS) and then coated with anti-MS2 antibody serving as receptor for the MS2 virus. Thus, a given layer of receptors may include the receptors only, or may include both the receptors and one or more types of binding agents or linker molecules.

Insofar as the grating 112 plays a role in the detection of binding events occurring at the receptors, the regions of the grating 112 containing the receptors may be considered to be parts of the respective sensors 254, 256. The grating 112 and sensors 254, 256 are configured such that irradiation of the sensors 254, 256 by an optical input beam 150 of a given wavelength λ (or band of wavelengths), and at a given coupling angle θ that fulfills the resonance condition, produces the guided mode beam 152. As schematically depicted in FIG. 2, the guided mode beam 152 may be considered as including N spatially distinct guided mode beam portions or components 262, 264 respectively propagating from the N sensors 254, 256. Depending on how the grating 112 is structured (unidiffractive or multi-diffractive), the guided mode beam components 262, 264 may propagate in one direction or in more than one direction along the waveguide plane. As also schematically depicted in FIG. 2, the optical output beam 132 outcoupled from the optical output edge 128 may be considered as including N spatially distinct optical output beam portions or components 266, 268 that correspond to the guided mode beam components 262, 264. The N sensors 254, 256 may be spaced from each other as needed for facilitating measurement of distinct output beam components 266, 268.

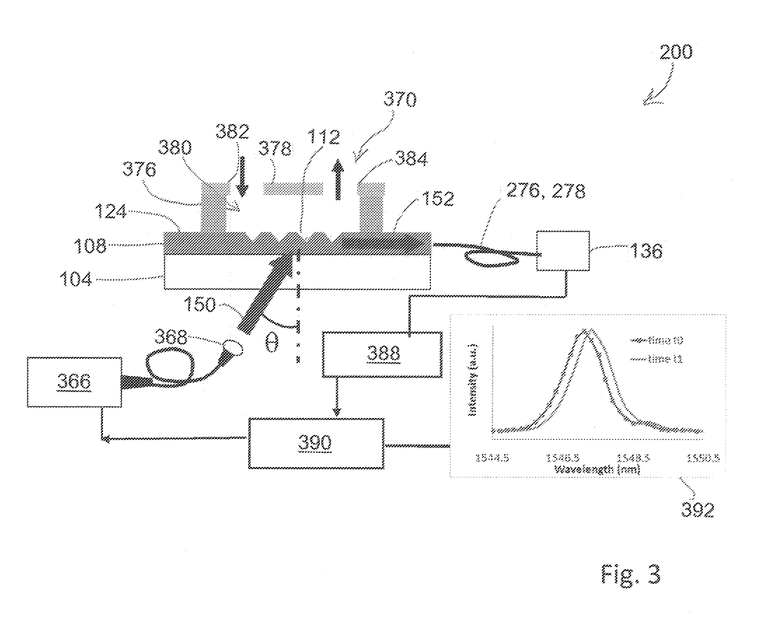

A collimated optical input beam 150 is generated by a suitable light source 366 (or photon source, FIG. 3). The light source 366 may be of the type that produces a diverging beam, such as a solid-state laser source (e.g., a VCSEL or DFB laser). A collimator lens may be utilized to collimate the diverging beam. The optical input beam 150 may be polarized or non-polarized. A linearly polarized beam may be obtained by positioning a linear polarizer (not shown) along the beam path or by utilizing a linearly polarized light source output. A linearly polarized beam may be conveniently aligned at a 45-degree angle relative to the planes of incidence so that both the TE and TM modes of the waveguide 108 can be coupled by the grating 112. Alternatively, the polarization of the linearly polarized beam may be may be oriented to select either TE or TM polarization such that the full light intensity is coupled into the selected guided mode.

In the present implementation, a single optical input beam 150 is expanded to simultaneously irradiate all sensors 254, 256 while remaining collimated. In the present implementation, a beam expander 270 of any suitable configuration may be provided to effect collimated, one-dimensional expansion of the optical input beam 150. The beam expander 270 may, for example, include a pair of cylindrical lenses. As appreciated by persons skilled in the art, a cylindrical lens has two curved faces that are cylindrical sections joined together at their edges such that the cross-section of the cylindrical lens is pillow-shaped. In the present implementation, the beam expander 270 is sized such that the width of the cross-sectional “line” of the optical input beam 150 spans the entire 1×N array of sensors 254, 256 whereby each sensor 254, 256 is simultaneously irradiated. Alternatively, beam collimation and expansion may be performed by a single optical element of suitable design. Alternatively, appropriate optics could be utilized to split the optical input beam 150 emitted from the light source 366 into multiple beams directed to individual sensors 254, 256. At present, however, one-dimensional expansion of a single optical input beam 150 is believed to be a more advantageous approach.

As noted above, the optical sensing device 100 produces multiple optical output beam components 266, 268 associated with the responses of the individual sensors 254, 256. The optical detector 136 includes a like number of optical detector units 272, 274 configured to receive the respective optical output beam components 266, 268. The optical detector units 272, 274 may be any devices configured for converting optical signals to electrical signals indicative of the intensities of the optical signals. For example, the optical detector units 272, 274 may be photodiodes, photocells, photomultipliers, or charge coupled devices (CCDs). The optical detector units 272, 274 may be the photo-sensitive elements of individual optical detectors 136, or may be part of the same optical detector 136 (e.g., a position-sensitive photodetector). The optical output beam components 266, 268 emitted from the optical output edge 128 of the optical sensing device 100 may be coupled in optical communication with the optical detector units 272, 274 by any suitable low-loss means. In the present implementation, optical fibers 276, 278 are positioned relative to the optical output edge 128 so as to efficiently collect the respective optical output beam components 266, 268. The optical fibers 276, 278 are connected to the respective optical detector units 272, 274. Either single-mode or multi-mode optical fibers 276, 278 may be utilized, with large-diameter multi-mode optical fibers being preferred in many implementations. Alternatively, optical fibers 276, 278 are not utilized and instead the optical detector units 272, 274 are positioned relative to the optical output edge 128 so as to directly receive the respective output beam components 266, 268. Thus, either the input ends of the optical fibers 276, 278 or the optical detector units 272, 274 themselves may be arranged in a 1×N array parallel to the 1×N array of sensors 254, 256.

FIG. 3 is a schematic view of an example of an optical sensing apparatus 200 that may include the optical sensing device 100 and associated components described above. In addition to the components described above, the optical sensing apparatus 200 includes a light source (photon source) 366. The light source 366 may be any source of electromagnetic radiation capable of emitting a beam of photons (optical input beam 150) at a wavelength λ suitable for the sensing technique disclosed herein, and capable of tuning the wavelength λ over a range of wavelengths near (above and below) its primary wavelength λ. Examples of the light source 366 may include various types of lasers, laser diodes (LDs) such as vertical-cavity surface-emitting lasers (VCSELs), frequency-swept lasers with external cavities, and light sources conventionally utilized in the optics-related fields of spectroscopy and spectrophotometry. In one non-limiting example, the light source 366 is an infrared (IR) wavelength-tunable laser such as a widely tunable distributed feedback (DFB) laser diode, such as the type conventionally utilized by the telecommunications industry in dense wavelength division multiplexing (DWDM) systems. This type of laser typically has wavelength-sweeping capability with wavelength spacing dictated by the ITU (International Telecommunication Union) grid. In some implementations the light source 366 is configured for emission at a principal wavelength of 1550 nm and is tunable over a range of 36 nm with a resolution of, for example, 25 GHz corresponding to 0.2 nm. The wavelength tuning range may, for example, include, fall within, or overlap with the C-band (1530 nm to 1565 nm) and/or the L-band (1565 nm to 1625 nm) utilized for optical telecommunications. The light source 366 may also be configured for emission at about 1310 nm. Other types of lasers that may be configured for large wavelength tunability include titanium-sapphire lasers. More generally, in some implementations the wavelength range over which the light source 366 is tunable may range from about 2 nm to about 250 nm. In other implementations, the tunable wavelength range is from about 30 nm to about 100 nm. Alternatively, the light source 366 may be configured for emission in other spectral ranges such as visible or near infrared.

The light source 366 includes a light source outlet 368 (e.g., a lens) from which the optical input beam 150 is emitted. The beam expander 270 (FIG. 2) is interposed between the light source outlet 368 and the optical sensing device 100. The light source outlet 368 and the beam expander 270, and/or the optical sensing device 100, may be mounted on a movable mechanical stage configured for orienting the optical input beam 150 at the desired coupling angle θ. Depending on the specific implementation, the coupling angle θ may range, for example, from −20 to 20 degrees, and more typically from −5 to 5 degrees, and in many implementations is preferably close to zero degrees, relative to the normal axis.

As also illustrated in FIG. 3, a fluidic structure 370 (which may be a microfluidic structure) is disposed on the optical sensing device 100 in a manner whereby the fluidic structure 370 encapsulates the grating 112 and the sensors 254, 256 without interfering with the outcoupling of optical signals from the waveguide 108. The fluidic structure 370 may be fabricated according to any suitable fabrication technique. The fluidic structure 370 may be fabricated separately from the optical sensing device 100 and thereafter clamped, adhered or otherwise attached to the optical sensing device 100. Alternatively, the fluidic structure 370 may be fabricated on the waveguide 108 as part of the process of fabricating the optical sensing device 100. In the present implementation, the fluidic structure 370 includes a spacer 376 disposed on the second surface 124 of the waveguide 108 and a cover or lid 378 disposed on the spacer 376. The components of the fluidic structure 370 may be composed of any material commonly utilized for microfluidic devices. In one non-limiting example, the spacer 376 is composed of silicone or polydimethylsiloxane (PDMS) and the lid 378 is composed of glass. The interfaces between the spacer 376 and the waveguide 108 and between the spacer 376 and the lid 378 may be fluid-tight with or without the need for additional seals such as gaskets or O-rings. The spacer 376 has a cavity that defines an internal chamber 380 of the fluidic structure 370. Hence, the internal chamber 380 is bounded laterally by the spacer 376, above by the lid 378, and below by the second surface 124 of the waveguide 108. The grating 112 and sensors 254, 256 are disposed in, and hence in fluid communication with, the internal chamber 380.

The lid 378 has a fluid inlet 382 and a fluid outlet 384 in fluid communication with the internal chamber 380. By this configuration, the fluidic structure 370 establishes a fluid flow path from the fluid inlet 382, through the internal chamber 380 (and in contact with the sensors 254, 256) and to the fluid outlet 384. The spacer 376 may be configured such that a common fluid flow path addresses all sensors 254, 256 together, or may provide multiple flow channels such that multiple flow paths address different sensors 254, 256, or different groups of sensors 254, 256, individually. The height (along the normal axis) of the internal chamber 380 or its flow channels may, for example, be 100 μm or less. The small volume provided by the internal chamber 380 enables a short binding reaction time (e.g., tenths of minute), which reduces the assay result time in comparison to the use of multi-well plates. The fluidic structure 370 also enables the use of a pump for controlled transport of fluids. Moreover, the fluid inlet 382 may include multiple inlet ports for communicating with different sources of fluids (e.g., fluid sample, wash/rinse solution, reagent, etc.). The fluid inlet 382 and fluid outlet 384 may, if desired, be configured for connecting to tubing in a conventional manner (e.g., Luer-type fittings). Examples of suitable inlet and outlet connectors include Nanoport™ connectors available from IDEX Corporation, Oak Harbor, Washington.

The fluidic structure 370 is configured to contain a fluid sample that serves as a low refractive-index upper cladding on the waveguide 108. The waveguide 108 maintains its ability to propagate a guided mode as long as the upper waveguide surface 124 is exposed to a material having a refractive index lower than that of the waveguide 108. The analyte solutions provided to the waveguide 108 by the fluidic structure 370 are typically aqueous (e.g., n˜1.33) and therefore adequately serve as an upper cladding. The spacer 376 of the fluidic structure 370 also serves as part of the upper cladding. Polymeric materials commonly utilized as the spacer 376 (e.g., silicone, PDMS) have refractive indices in the range of 1.4 to 1.5, and other polymers suitable for use as spacers 376 may have refractive indices as high as 1.6. All such materials may be utilized in conjunction with waveguide films of a higher refractive index. Alternatively an upper cladding film (not shown) may be deposited on the waveguide surface 124 with an opening on the grating 112. The properties of the upper cladding film may be the same as the interlayer 114 to make the optical sensing device 100 optically compatible with all possible fluidic structures 370. It is, however, more convenient in many implementations to avoid the use of a dedicated upper cladding film.

The optical sensing apparatus 200 may further include a digitizer 388, an electronic processor-based controller (electronic controller) 390, and a user output device 392. The digitizer 388 is in signal communication with the optical detector 136. The digitizer 388 may be any device suitable for converting the analog measurement signals received from the optical detector 136 to digital measurement signals, which facilitates further processing and analysis of the measurement signals. In typical implementations the digitizer 388 is a digital acquisition (DAQ) card, the general operation of which is familiar to persons skilled in the art. The electronic controller 390 is in signal communication with the digitizer 388 for receiving the digital measurement signals and controlling data acquisition. The electronic controller 390 is configured for processing the signals to provide useful data to a user regarding analyte binding events detected by the optical sensing device 100. The electronic controller 390 is configured to output the data to the user output device 392 in format that enables the user output device 392 to present the data in a readily understandable format. By way of example, the user output device 392 is illustrated in FIG. 3 as displaying a wavelength shift (intensity as a function of wavelength at two time points t₀ and t₁), which may be indicative of a specific analyte binding event and consequently indicative of the presence of the specific analyte in the fluid sample flowed through the fluidic structure 370. In the present implementation, the electronic controller 390 is also in signal communication with the light source 366 to control its operation, e.g., on/off operation as well as wavelength scanning or sweeping (e.g., the rate of increase/decrease). The electronic controller 390 may also control other components of the optical sensing apparatus 200 such as, for example, a pump (not shown) that establishes the flow of the fluid sample through the fluidic structure 370. For all these purposes the electronic controller 390 may include hardware, firmware, software, etc. as needed, as appreciated by persons skilled in the art. Software utilized for controlling data acquisition may provide a graphical user interface (GUI), one example of which is LabView® software. The user output device 392 may be any read-out or display device and thus include, for example, a liquid crystal display (LCD) screen and/or a printer. As also appreciated by persons skilled in the art, the electronic controller 390 may also communicate with one or more types of user input devices (keypad, switches, buttons, touch screen, etc.).

The optical sensing device 100 generally operates as a refractometer, but with the grating 112 functionalized more specifically operates as a biochemical sensing device. In operation, the light coupled into the waveguide 108 of the optical sensing device 100 by the grating 112 is extremely sensitive to the coupling angle θ and the refractive index of the media above the grating (i.e., the layers of receptors and the fluid sample). The occurrence of an adsorption event such as an analyte binding to a receptor modifies the refractive index of the media above the grating 112. Modification of the refractive index has the effect of shifting the resonance condition (coupling condition) of the optical sensing device 100. The resonance condition may be represented by the coupling equation for an m-order diffraction linear grating in air: N_{eff} (n_{core}, t, λ)=sin(θ)+mλ/Λ, where N_{eff} is the effective index of the waveguide mode and depends on the index of refraction n_{core} of the waveguide 108, the thickness t of the waveguide 108 and the wavelength λ of the light, and where m=±1 or ±2 is the diffraction order. A shift in the resonance condition may be detected either as an angular shift Δθ or a wavelength shift Δλ. Measurements based on angular interrogation would require costly and relatively complex hardware for changing the orientation of the optical input beam 150 relative to the optical sensing device 100 during testing. Instead, the present implementation takes measurements based on swept wavelength interrogation. In this way, the relative positions of the optical input beam 150 and the optical sensing device 100 are fixed at a given coupling angle θ, the wavelength λ of the optical input beam 150 is swept (scanned), and consequently adsorption events are detected as shifts in wavelength Δλ.

As an example, FIG. 2 illustrates a display 280 of measurement data derived from processing the outcoupled beam component 266 corresponding to the guided mode component 262 propagating from the binding-specific sensor 254. Specifically, the measurement data are plots of intensity (arbitrary units) as a function of wavelength (nm). A wavelength-sweeping measurement provides a spectral curve which contains an intensity peak related to the resonance condition and sharp drops in measured intensity on either side of the peak, corresponding to off-resonance wavelengths for which the optical input beam 150 passes through the optical sensing device 100 without being coupled into the waveguide 108 as a guided mode. The resonance wavelength position for a curve may be accurately determined with a variety of data-processing algorithms including centroid methods and regression curve fit. In the example of FIG. 2, the measurement data contain a first resonance curve 282 acquired at a first point in time t₁ and a second resonance curve 284 acquired at a second point in time t₂. For instance, the first curve 282 and the second curve 284 may be generated by flowing a fluid sample through the fluidic structure 370 of the optical sensing device 100 and into contact with the binding-specific sensor 254, and taking optical readings at times t₁ and t₂. The resonance wavelength for the first curve 282 is λ₁ and it is different and larger than wavelength λ₂ for the second curve 284. The difference between the resonance wavelength λ₁ and λ₂ is indicative of a wavelength spectral shift (i.e., a shift in the value of the resonant wavelength) which is caused by a change in the index of refraction in the fluid sample at (or near, i.e., just above) the waveguide surface at the binding-specific sensor 254. Because the sensor 254 is coated with receptors specific to the analyte, the wavelength shift may be interpreted as the presence of the target analyte in the fluid sample.

In the present implementation a multi-channel configuration, in which at least one reference sensor 256 is provided, addresses the contingency that non-specific binding or instrumental drift may occur, which needs to be distinguished from the specific-binding related to the presence of analyte in the sample. As an example, FIG. 2 further illustrates a display 290 of reference data derived from processing the optical output beam component 268 corresponding to the guided mode component 264 propagating from the reference sensor 256. In this example, the reference data contain a first curve 292 acquired at a first point in time t₁ and a second curve 294 acquired at a second point in time t₂. The second curve 294 is shifted from the first curve 292. This wavelength spectral shift may be indicative of the occurrence of a non-specific adsorption event. For instance, the fluid sample may contain non-analyte components which adventitiously adsorb to the grating surface from mechanisms others than specific recognition. A non-analyte component bound to the reference sensor 256 changes the refractive index and causes a wavelength spectral shift which may be interpreted as the occurrence of a binding event on the reference sensor 256. Alternatively, the wavelength spectral shift may be indicative of instrumental drift. The response of the reference sensor 256 to the testing of the fluid sample thus may be utilized as a baseline for the test and to correct for instrumental drifts. The electronic controller 390 of the optical sensing apparatus 200 may be configured, for example, to subtract the response signal generated by the reference sensor 256 from the response signal generated by the binding-specific sensor 254, thereby eliminating the influence of non-specific binding events and/or instrumental drifts from the assay being performed.

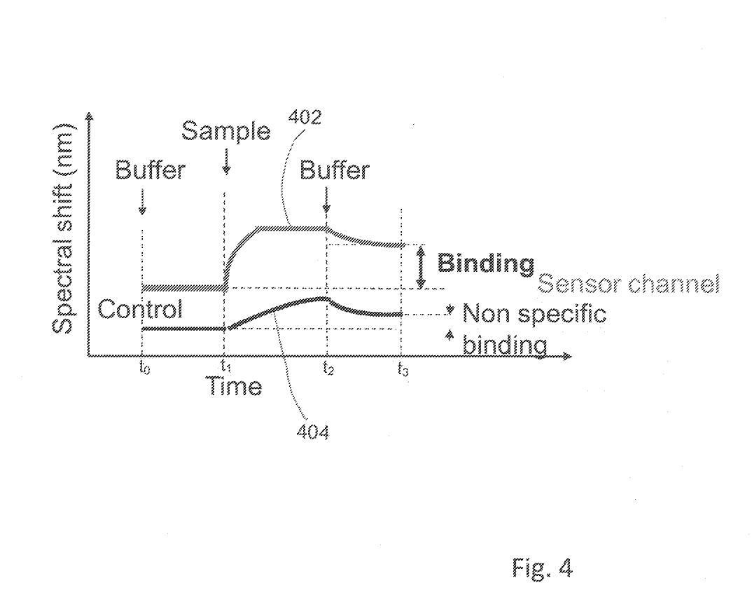

FIG. 4 is an example of data processed from multiple measurement data obtained by the optical sensing apparatus 200. FIG. 4 is illustrative of an example of performing a direct binding assay. It will be understood, however, that the optical sensing device 100 and associated optical sensing apparatus 200 may be configured appropriately for performing other types of assay formats such as, for example, sandwich assays, competitive assays, and inhibition assays. FIG. 4 includes a signal 402 derived from the output signal of a binding-specific sensor 254, and a signal 404 derived from the output signal of a reference sensor 256. The signals 402, 404 are plots of wavelength spectral shift (nm) as a function of time. From time t₀ to time t₁ only a reference solution is flowed through the fluidic structure 370 of the optical sensing device 100, thereby producing flat signals 402, 404 in both the binding-specific and reference channels and indicating the non-occurrence of any binding events. The reference solution is selected for having a refractive index as similar as possible to the fluid sample to reduce the effect of volumetric sensing. A flow of a fluid sample through the fluidic structure 370 is initiated at time t₁ and continued until time t₂. During this time, target analytes in the fluid sample become bound to the analyte-specific receptors of the binding-specific sensor 254, and other components of the fluid sample may become bound to the reference receptors of the reference sensor 256. The accumulation of these bound analytes and other components is evident from the rises in the respective signals 402, 404, indicating an increasing concentration of the bound analytes and other components on the respective binding-specific sensor 254 and reference sensor 256 over time. At time t₂ the flow of the fluid sample is replaced with another flow of the buffer solution. Detector readings taken from time t₂ to time t₃ indicate a slight reduction in the concentrations of both the bound analytes and the bound non-analyte components. This may be indicative of some analytes and non-analyte components being not tightly bound to the functionalized layer and being removed by the flow of the buffer solution. In further processing, the reference signal 256 may be subtracted from the binding-specific signal 254 to produce a single signal accurately indicative of target analyte binding events and providing a quantitative measurement of the target analyte mass or concentration target analytes.

The optical sensing apparatus 200 described above may be advantageously implemented as a portable instrument. In such implementation, the optical sensing apparatus 200 may include a portable housing (e.g., an enclosure, module, or the like) in which some or all of the various components of the optical sensing apparatus 200 may be mounted in a suitable manner. The user input devices (not shown) and user output devices 392 may be located on one side of the housing or at a console area of the housing. The optical sensing apparatus 200 may include a battery or other internal power source located in the housing, and/or may be include a port configured for connection with an external power source. The pump utilized to move fluid samples through the fluidic structure 370 (e.g., a peristaltic pump, syringe pump, etc.) may be located internal or external to the housing. The housing may include inlet and ports for routing fluid samples, reagents, wash/rinse buffers, and the like through the fluidic structure 370. The housing may also include one or more conventional signal communication ports for communicating with external computing devices (e.g., laptop, personal digital assistant, etc.), external input peripherals, external output peripherals, etc. The use of the input/output interfaces of an external computing device may eliminate the need for providing user input and output devices 392 with the housing.

Moreover, the housing may be configured such that the optical sensing device 100 (alone or with the fluidic structure 370) is installable in and removable from the housing as a modular component. In this manner, the optical sensing device 100 may be provided as a disposable (single-use) device, thereby facilitating safe disposal of the used optical sensing device 100 and lowering the risk of contamination or infection from the analytes tested. For some analytes and certain assays, the optical sensing device 100 may be reusable after appropriate regeneration of the functionalized surface. For instance, as appreciated by persons skilled in the art, many types of ligands and other types of binding partners may be removed from surface-immobilized receptors by applying a high- or low-pH solution or cleaving enzymes such as pepsin, without significantly affecting the binding capability of the functionalized surface. The modular configuration also enables the selection of different optical sensing devices 100 to be utilized with the optical sensing apparatus 200. For instance, different optical sensing devices 100 may be functionalized with different types of binding-specific receptors and thus configured for different types of assays.

As an example of a modular configuration, the housing may include a sample holder (e.g., a bay) configured to receive and secure a cartridge in which the optical sensing device 100 (with the fluidic structure 370 attached thereto) is mounted. The housing may be configured such that upon installing the cartridge into the sample holder, the fluid inlet 382 and fluid outlet 384 of the fluidic structure 370 are respectively coupled in a sealed manner with a fluid input port and a fluid output port of the housing. For example, inlet tubing leading from a container containing the fluid sample to be analyzed may be connected to the fluid input port, and outlet tubing leading to a waste receptacle may be connected to the fluid output port. The pump may be located in-line with either the inlet tubing or the outlet tubing to either push or pull the liquid sample through the fluidic structure 370.

According to other implementations, the present disclosure provides various kits for carrying out the optical sensing techniques described herein. In some implementations, a kit may include a set of optical sensing devices 100. All of the optical sensing devices 100 of the kit may be configured to perform the same assay. Alternatively, the kit may include optical sensing devices 100 configured for performing different assays. The optical sensing devices 100 of the kit may be disposable for single-use assays. Alternatively, the optical sensing devices 100 of the kit may be configured for reuse. The kit may include tools and reagents as needed for regenerating the sensor surfaces of the optical sensing devices 100. The optical sensing devices 100 of the kit may be preconfigured for performing particular assays. Alternatively, the kit may include tools and reagents as needed for enabling a user to configure the optical sensing devices for different assays. In other implementations, a kit may include one or more optical sensing devices 100, and also an optical sensing apparatus 200 configured for use with the optical sensing devices 100. The optical sensing apparatus 200 of the kit may include one or more components as described above and illustrated in FIGS. 2 and 3. The optical sensing apparatus 200 of the kit may be provided in the form of a portable housing that is ready for coupling with various external components such as, for example, fluid handling components, a portable computing device, or the like.

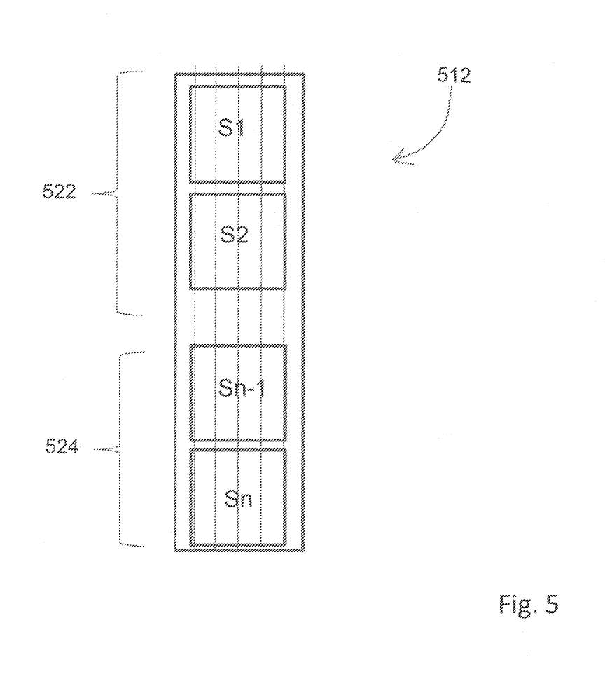

FIG. 5 is a schematic top plan view of an example of a diffraction grating 512 with N sensors, e.g., S₁, S₂, . . . , S_{N-1}, and S_{N}. As in other implementations described herein, all N sensors may be arranged in a one-dimensional array and capable of producing N individually distinguishable guided mode beam components that propagate to the designated optical output edge of the waveguide, from which the corresponding output beam components may be coupled into separate optical detector units for measurement and subsequent signal processing. Generally, the sensors may include one or more binding-specific sensors. In many implementations, the sensors may additionally include one or more reference sensors. A reference sensor is typically positioned adjacent to a corresponding binding-specific sensor to provide a reference measurement in conjunction with the target measurement derived from the binding-specific sensor. In the case of multiple binding-specific sensors, at least one binding-specific sensor may have a different configuration than one or more of the other binding-specific sensors. For example, sensor S₁ may be a first binding-specific sensor containing receptors of a first type, while sensor S_{N-1} may be a second binding-specific sensor containing receptors of a second type different from the first type. As a specific example, the receptors of sensor S₁ may be antibodies having an affinity for a certain antigen, while the receptors of S_{N-1} may be different antibodies having an affinity for a different type of antigen. The two different antigens may be associated with the same class of infection for which diagnosis is being sought. For instance, the two different antigens may be associated with two different serotypes of the same type of virus. Alternatively, the receptors of sensor S₁ may be different than the receptors of S_{N-1} but both types of receptors have an affinity for the same antigen. In some implementations, some of the binding-specific sensors may have the same configuration (type of receptors).

Certain sensors may be associated with each other as distinct groups or sets of sensors (sensor sets). For example, a first sensor set 522 may include sensors S₁ and S₂, a second sensor set 524 may include sensors S_{N-1} and S_{N}, and additional sensor sets (not shown) may be included between the first sensor set 522 and the second sensor set 524 along the 1×N array. Each sensor set 522, 524 may be configured for carrying out a different assay or a different aspect of a particular assay. Different sensor sets 522, 524 may be utilized (irradiated and exposed to fluids) simultaneously or sequentially according to a predetermined procedure, such as a procedure requiring different assay operations for different target analytes. For example, in a sandwich assay the first sensor set 522 may test for a first type of antigen associated with a certain type of infection using a certain amplification solution after analyte binding has occurred. In this same sandwich assay the second sensor set 524 may test for a second type of analyte, for example an antibody associated with the same type of infection but requiring a different amplification solution than that utilized for the first sensor set 522. When it is desired to provide different sensor sets 522, 524 to serve different functions as in the examples just noted, the fluidic structure attached to the optical sensing device may be configured to provide different flow channels, thereby in effect defining the different sensor sets 522, 524 which respectively communicate with the different flow channels. In some implementations, each sensor set 522, 524 may include at least one reference sensor for that particular sensor set 522, 524. For example in the first sensor set 522 illustrated in FIG. 5, sensor S₁ may be a binding-specific sensor specific to a first type of analyte and sensor S₂ may be a reference sensor, while in the second sensor set 524 sensor S_{N-1} may be a binding-specific sensor specific to a second type of analyte and sensor S_{N} may be a reference sensor. Moreover, a given sensor set 522, 524 may include more than one binding-specific sensor (not shown). One or more of the binding-specific sensors of the same sensor set 522 or 524 may be configured differently (have different receptors) than the other binding-specific sensors of the same sensor set 522 or 524.

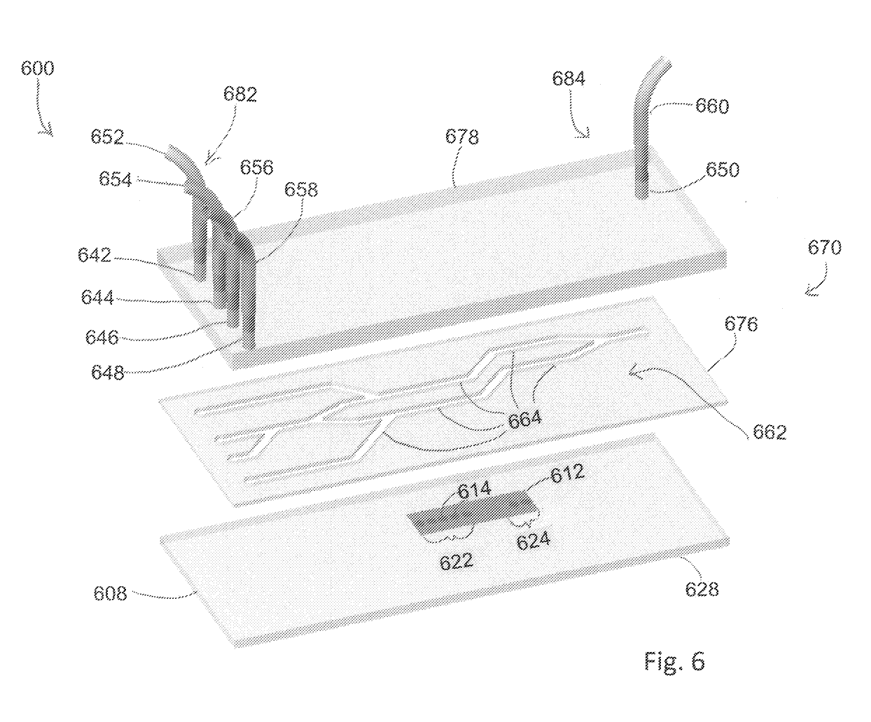

FIG. 6 is an exploded view of an example of an optical sensing device 600 that includes a multi-channel fluidic structure 670. For instance, the optical sensing device 600 and fluidic structure 670 illustrated in FIG. 6 may be utilized with the optical sensing apparatus 200 illustrated in FIG. 3. As described above, the optical sensing device 600 includes a waveguide 608 (and underlying substrate), a diffraction grating 612 disposed on a surface of the waveguide 608, and a one-dimensional array of sensors 614 disposed on the grating 612. The grating 612 may include grooves (not shown) or another suitable periodic structure. The grooves and the sensors 614 may be arranged in parallel with an optical output edge 628 of the waveguide 608. In the present implementation, the fluidic structure 670 is configured such that the fluid flow is parallel to the groove direction, while in other implementations the fluidic structure 670 may be configured such that the fluid flow is perpendicular to the groove direction. In the present implementation, the longer side of the waveguide 608 serves as the optical output edge 628, while in other implementations the shorter side may serve as the optical output edge 628. In the present implementation, the sensors 614 are grouped as a first sensor set 622 and a second sensor set 624. The first sensor set 622 includes four sensors 614 and the second sensor set 624 includes two sensors 614, although it will be understood that any of the sensor sets 622, 624 may include any number of sensors 614 as needed for the particular assay(s) contemplated for the optical sensing device 600. In some implementations, at least one of the sensors 614 of the first sensor set 622 may be a reference sensor, and at least one of the sensors 614 of the second sensor set 624 may be a reference sensor.

As described above, the fluidic structure 670 includes a spacer 676, a lid 678, a fluid inlet 682 and a fluid outlet 684. In this example, the fluid inlet 682 includes four separate inlet ports 642, 644, 646, 648 for introducing flows of four different fluids into the fluidic structure 670, and a single common outlet port 650 for routing all fluids to a waste receptacle or other desired destination. This configuration enables the flow of multiple fluids to be driven by aspiration at the common outlet port 650 and hence avoids the need for multiple fluid pumps or a bulky multi-channel pump. The inlet ports 642, 644, 646, 648 may be connected to respective fluid conduits 652, 654, 656, 658 (e.g., tubing) for receiving fluids from four separate sources (e.g., vials, reservoirs, etc.), and the outlet port 650 may be connected to a fluid conduit 660 leading to a waste receptacle. As an example, the first inlet port 642 may be configured for flowing a first reagent to the first sensor set 622, the second inlet port 644 may be configured for flowing a buffer solution to both the first sensor set 622 and the second sensor set 624, the third inlet port 646 may be configured for flowing a fluid sample (potentially containing target analytes) to both the first sensor set 622 and the second sensor set 624, and the fourth inlet port 648 may be configured for flowing a second reagent to the second sensor set 624. The reagents may be any solutions providing a function specific to the test associated with a particular sensor set 622, 624. As examples, the reagents may facilitate a particular test (such as an amplification reagent configured to amplify the measurement signal), or may be a required additive of a particular assay format (such as in an inhibition assay, where one reagent may be a solution of analyte conjugates and another reagent may be a solution of analyte conjugates reacted with the fluid sample). A variety of buffer solutions may serve a variety of purposes, such as washing/rinsing and/or providing a baseline measurement (e.g., phosphate buffered saline or PBS), or removing bound molecules from receptors to regenerate the sensor surfaces (e.g., acid solution).

The binding-specific sensors of the first sensor set 622 may be configured differently than the binding-specific sensors of the second sensor set 624, and thus the first reagent may be different from and incompatible with the second reagent and the flows of the two reagents are not to be mixed before the optical sensing measurement. For these purposes, in this example the spacer 676 is configured to provide a network 662 (plurality) of flow channels. The flow channels are defined by a plurality of different flow channel sections, a few of which are designated 664 as examples. Four flow channel sections communicate with the respective inlet ports 642, 644, 646, 648, and one flow channel section communicates with the common outlet port 650. Intermediate flow channel sections conduct different fluid flows to the sensor sets 622, 624. Some intermediate flow channel sections may merge together or split apart as needed for merging or splitting various flows.

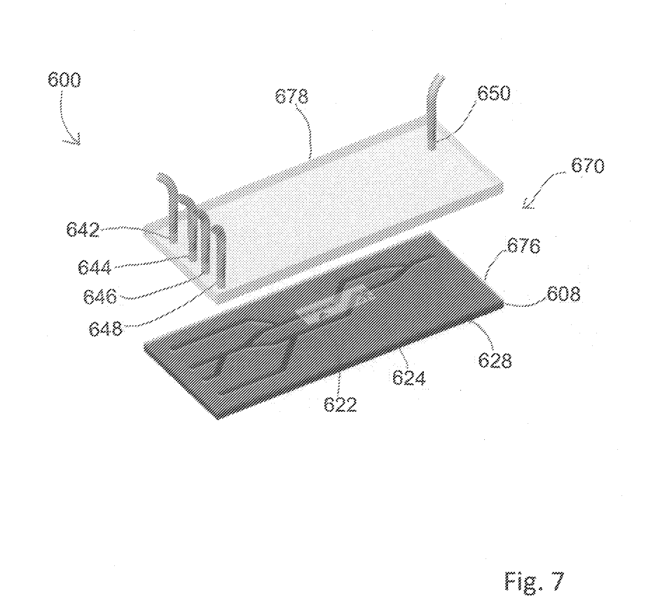

FIG. 7 is another exploded view of the optical sensing device illustrated in FIG. 6, but with the spacer 676 disposed on the surface of the waveguide 608. It can be seen that in this example, the fluidic structure 670 when assembled on the waveguide 608 establishes four primary flow channels. The first flow channel conducts a first reagent from the first inlet port 642 to the first sensor set 622, and to the outlet port 650. The second flow channel conducts a buffer solution from the second inlet port 644, to the first sensor set 622 and the second sensor set 624, and to the outlet port 650. The third flow channel conducts a fluid sample from the third inlet port 646, to the first sensor set 622 and the second sensor set 624, and to the outlet port 650. The fourth flow channel conducts a second reagent from the fourth inlet port 648 to the second sensor set 624, and to the outlet port 650. Two or more of the flow channels may share one or more of the flow channel sections. In the present example, the flow channel sections are arranged such that the flow channel for the buffer solution merges with the flow channel for the fluid sample, and subsequently these two flow channels split into two separate branches that lead to the respective first sensor set 622 and second sensor set 624. Also, the flow channel for the first reagent merges with the flow channel for the buffer/fluid sample at a point upstream of the first sensor set 622. The flow channel for the second reagent merges with the other flow channel for the buffer/fluid sample at a point upstream of the second sensor set 624. The flow channels leading from the two sensor sets 622, 624 merge into a common flow channel that leads to the output port.

Other implementations for bringing fluid samples into contact with the sensors can be envisioned. Various other suitable geometrical arrangements of flow patterns over the sensors may be implemented. For example, the flow pattern may provide one flow channel per sensor if the flow is arranged orthogonally to the direction of the grooves.

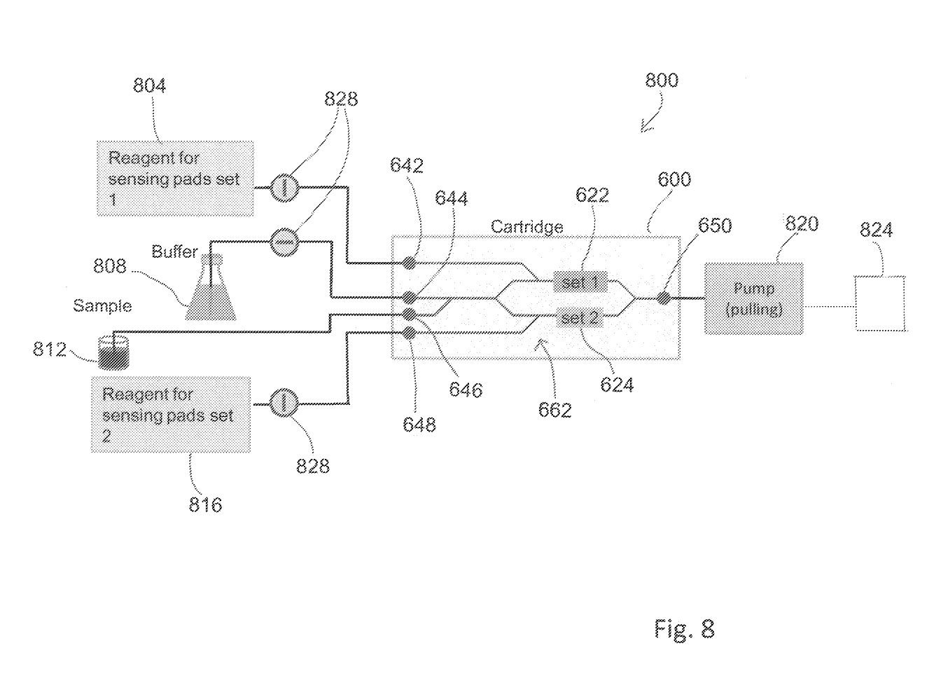

FIG. 8 is a schematic view of an example of a fluid system 800 in which an optical sensing device 600 and fluidic structure 670 such as illustrated in FIGS. 6 and 7 may operate. As appreciated by persons skilled in the art, some or all of the components of the fluid system 800 may be provided in a housing of the optical sensing apparatus 200, or some of the components may be coupled to the housing via fluid connections of the optical sensing apparatus 200. For the convenience of the schematic illustration, the sensor sets 622, 624 are not shown as being aligned in the one-dimensional array shown in FIGS. 6 and 7. In addition to the optical sensing device 600 and the fluidic structure 670 coupled thereto, the fluid system 800 may include a plurality of separate fluid sources communicating with different inlet ports 642, 644, 646, 648. Continuing with the present example, the fluid system 800 may include a first reagent source 804 communicating with the inlet port 642, a buffer solution source 808 communicating with the inlet port 644, a sample source 812 communicating with the inlet port 646, a second reagent source 816 communicating with the inlet port 648, a fluid moving device 820 (e.g., pump or the like) communicating with the outlet port 650, and a fluid collection receptacle or destination 824 communicating with the fluid moving device 820, all via respective fluid lines (e.g., tubing, conduits or the like). The fluid system 800 may further include fluid flow control devices 828 operatively communicating with one or more of the fluid lines, such as valves. The fluid flow control devices 828 may be passive devices (e.g., check valves) or actively controlled devices (e.g., pinch valves, solenoid valves, etc.). The fluid flow control devices 828 and the fluid moving device 820 may be manually operated, or they may be automated such as via control signals from the electronic controller 390 of the optical sensing apparatus 200. The fluid system 800 of the present example is configured such that only one fluid moving device 820 is required, which may result in a more compact and cost-effective configuration as compared to requiring independent flow actuation for each separate flow channel.

Depending on the assay steps or other factors, the fluid moving device 820 (and fluid flow control devices 828, if provided) may be operated to bring a fluid sample into contact with the sensors 614 in different ways. As examples, fluid flow may be stopped over the sensors 614 to allow for a reaction or incubation time. Fluid flow may be either stopped over the sensors 614 or allowed to proceed at a controlled (typically slow) flow rate during the taking of optical readings.

The optical sensing devices described in the present disclosure may be configured in different ways to enable a variety of assay formats. Such assay formats include, but are not limited to, direct binding assays, sandwich assays, competitive assays, and inhibition assays. As appreciated by persons skilled in the art, the type of assay format will determine the type of receptors immobilized on the waveguide to provide sensors, the functions served by the binding-specific sensors and reference sensors, the types of reagent and buffer solutions utilized, and the particular steps required for carrying out the assay format.

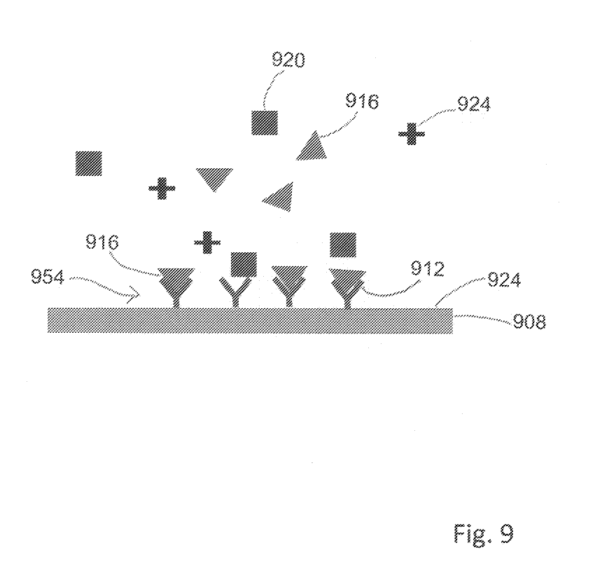

FIG. 9 illustrates an example of a direct binding assay. Specifically, FIG. 9 is a schematic view of an example of a waveguide 908 that includes a binding-specific sensor 954. The binding-specific sensor 954 is formed by immobilizing a plurality of binding-specific receptors 912 (i.e., forming a layer of binding-specific receptors 912) on a diffraction grating (not specifically shown) disposed on an upper surface 924 of the waveguide 908. A fluid sample has been brought into contact with the binding-specific sensor 954. As schematically represented in FIG. 9, the fluid sample contains analytes 916 for which detection is sought as well as various types of non-analyte components such as non-analyte components 920 and 924. It can be seen that when the binding-specific sensor 954 is exposed to the fluid sample, the analytes 916 become captured by the binding-specific receptors 912 while the non-analyte components 920 and 924 do not. These binding events may be detected and the mass or concentration of the bound analytes 916 determined in the manner described above. Prior to flowing the fluid sample to the binding-specific sensor 954, a buffer solution of a predetermined composition may be flowed to the binding-specific sensor 954 and optical readings taken to provide a baseline measurement. Additionally, the same fluid sample may be flowed (typically simultaneously) to an appropriately configured reference sensor (not shown) to provide or contribute to the baseline measurement. The reference sensor may, for example, include receptors capable of binding to the non-analyte components 920 and 924. Subtraction of the measurements provided by the reference sensor from the measurements provided by the binding-specific sensor 954 may ensure that non-analyte binding events are not interpreted as analyte binding events.

As appreciated by persons skilled in the art, in other assay formats not entailing the binding of analytes 916 directly to the immobilized receptors 912, the receptors 912 may be configured to bind specifically to non-analyte binding partners. The role played by these captured non-analyte binding partners in the determination of the presence of the analytes 916 in the fluid sample depends on the particular assay being carried out (e.g., competitive assay, inhibition assay, etc.).

The optical sensing devices described herein exhibit a high enough sensitivity to changes in refractive index, a high enough specificity and affinity to targeted binding partners, and a low enough limit of detection (LOD) that direct binding assays will produce signals sufficient for diagnostic testing of many types of analytes.

Another way of enhancing the signal, sensitivity and LOD is to perform a sandwich assay. In this format, the surface-immobilized receptors are binding-specific receptors having a specific affinity for the analytes sought to be detected in the fluid sample. As with a direct assay, the fluid sample is flowed into contact with the sensors of the optical sensing device, and analytes in the fluid sample are provided the opportunity to be captured by the receptors. Subsequently, a reagent solution containing secondary binding partners is flowed into contact with the sensors. Like the receptors, the secondary binding partners are specific to the analyte. The secondary binding partners and the receptors may be the same molecules or different molecules. For example, in a case where the analyte to be detected is an antigen, the receptors may be an antibody against the analyte, and the secondary binding partners of the reagent solution may also be an antibody against the same analyte. The antibodies utilized as the secondary binding partners may be the same antibodies as those utilized as the receptors or different antibodies. If analytes have been captured by the receptors, then secondary binding partners of the added reagent solution will bind to the captured analytes (i.e., creating a receptor-analyte-secondary binding partner “sandwich”). The binding of the secondary binding partners to the analytes results in a detectable change in refractive index, which may be more pronounced and easier to detect as compared to just the binding of the analytes to the immobilized receptors of the sensors.

Depending on the assay to be performed, a competitive or inhibition assay format may be preferable to the direct binding or sandwich assay format, such as when the analyte of interest is small (e.g., MW<5000) and thus may not produce a sufficiently large change in refractive index when captured by the receptor serving as a specific binding partner.

In a competitive assay, as in direct binding and sandwich assays, the surface-immobilized receptors are binding-specific receptors having a specific affinity for the analytes sought to be detected in the fluid sample. A secondary binding partner is added to the fluid sample. In this case, the secondary binding partner is a large molecule (relative to the analyte) presenting one or more binding sites having an affinity for the analyte. Hence, interaction between the analyte and the secondary binding partner produces conjugates of the analyte (complexes in which the analyte is conjugated with the secondary binding partner). The secondary binding partner is added to the fluid sample by allowing the fluid sample to be incubated with the secondary binding partner for a period of time prior to introduction of the fluid sample to the optical sensing device, or by adding a solution containing pre-formed conjugates to the fluid sample. In either case, when the fluid sample containing the conjugates is flowed to the sensors of the optical sensing device, the conjugated analytes compete with any “free” analytes in the fluid sample (i.e., the pre-existing analytes, if any, whose presence is unknown and sought to be detected) for the limited number of binding sites (the surface-immobilized receptors) presented by the sensors. Unlike the cases of direct binding and sandwich assays, the signal measured in response to the binding events is inversely proportional to the concentration of the free analytes in the fluid sample.

In an inhibition assay, the immobilized receptors may be the same type of analytes sought to be detected in the fluid sample, or may be complexes formed with these analytes. The fluid sample is prepared by adding a predetermined amount of a binding partner specific to the analyte of interest. The analytes of interest, if present in the fluid sample, bind to the binding partners. The fluid sample containing the as-formed analyte-binding partner complexes is then flowed to the sensors of the optical sensing device. Binding partners of the fluid sample not already bound to analytes of the fluid sample are captured by the immobilized analyte receptors of the sensors. Thus, like a competitive assay, the signal measured in response to the binding events is inversely proportional to the concentration of the free analytes in the fluid sample.

Additional examples of various surface functionalization techniques and assay detection formats, and their applications for detection/diagnosis in various medical, chemical and biological contexts, are described in J. Homola, Surface Plasmon Resonance Sensors for Detection of Chemical and Biological Species, Chem. Rev., Vol. 108, 462-493 (2008), which is incorporated by reference herein in its entirety.

The optical sensing device according to various implementations disclosed herein operates as an optical evanescent wave sensor to interact with analytes bound to the waveguide surface. The optical sensing device thus is label-free, e.g., it does not require the use of fluorophores or chemiluminescent probes, and enables analyte detection in rapid fashion. Moreover, the optical sensing device has micro-scale features and thus may be microfabricated in a cost-effective manner and may be utilized in conjunction with cost-effective, compact optical read-out apparatus. Additionally, with the input grating-coupler configuration and edge detection (out-coupling from the optical output edge), the optical sensing device requires only one grating and in particular does not require an output grating-coupler. This configuration reduces the complexity, cost and footprint of the optical sensing device and associated apparatus or system. This configuration also eliminates the problem of spurious reflection to the detector, contrary to gratings operating in the reflection mode. Also, because a typical assay performed by the optical sensing device only requires a few steps and a small fluid volume (e.g., about 100 μL) to detect binding events, the assay may be performed in a few minutes and in an automated fashion, and further does not require complex laboratory work.

Moreover, the use of a largely tunable light source facilitates the use of wavelength interrogation as opposed to angular interrogation. This configuration relaxes the requirements for precise mechanical alignment and positioning as between the light source and the optical sensing device. For implementations entailing the insertion of the optical sensing device into a housing of the optical sensing apparatus, this configuration allows tolerance in the mechanical alignment associated with the insertion. The angular (and therefore wavelength) acceptance of the grating is narrow by design because the grating serves both as the light coupler as well as the sensor (when functionalized) in this configuration. This narrow angular acceptance of the grating coupler makes the alignment of the optical sensing device with respect to the laser beam critical. Previously known approaches utilize multiple gratings for sensing, and adopt for convenience a large coupling angle tolerance designed for the input grating coupler while the other gratings are utilized for sensing. In the optical sensing device of the present disclosure in which one grating serves as both the light coupler and the sensor, and utilizing an infrared (e.g., 1550 nm) wavelength and a grating with for example a 1000-nm pitch, the relationship between a change in angular alignment and a wavelength shift in the optical signal may be calculated as Δλ (nm)≈18 Δθ (deg) for a coupling angle θ near zero. Thus, for example, a misalignment of 0.1 angular degree causes a signal shift of 1.8 nm, which is well within the range of light sources of the type contemplated for the presently disclosed implementations such as lasers commonly utilized in DWDM telecommunications. Such lasers provide size and cost advantages and are subject to ongoing improvement by the telecom industry. The use of such lasers is enabled by waveguides exhibiting high optical transmission efficiency and therefore low propagation losses in the near infrared where the telecommunications industry operates (e.g., a wavelength range of about 0.8 to 1.7 μm). The optical transmission properties of waveguides of the type described herein are controllable through deposition conditions, and the material composition of such waveguides is compatible with the wavelengths emitted by such lasers (e.g., about 1550 nm). Additionally, recently developed DWDM lasers have a wavelength spacing (or step) as low as 0.2 nm. Such a wavelength spacing is adequate for probing the relatively broad coupling peak (typically 1-2 nm) of the gratings disclosed herein. By contrast, other optical sensor technologies such as those based on ring resonators have much narrower coupling peaks (0.1-0.2 nm) and require higher resolution wavelength-sweeping lasers and their attendant increased cost and bulkiness.

The use of a wavelength-tunable light source emitting at relatively long wavelengths in conjunction with the optical sensing device according to the present teachings provides unexpected benefits. Surprisingly, such a light source enables the optical sensing device to achieve high sensing performance in conjunction with various (bio)chemical and immunological assays. Additionally, the light source of this type can be provided in a compact form and is highly suited for integration in a portable optical sensing apparatus. Additionally, as a light source of this type has been conventionally utilized in telecommunications as noted above, it is readily available, cost-effective, and subject to ongoing improvements in optical performance, reliability and ruggedness.

### EXAMPLE

The following Example describes the fabrication and evaluation of an optical sensing device and associated apparatus in which the optical sensing device has an input grating-coupler configuration with functionalized sensor areas as described above. This Example demonstrates the utility of the optical sensing device as an optical evanescent wave sensing platform for performing label-free assays, utilizing wavelength interrogation in the telecommunications spectral range as the transduction mechanism. Evaluation of the optical sensing device demonstrated that high-performance volumetric sensing can be achieved with the use of a compact, low-cost telecommunications laser. The footprint of the system described in this Example (including the light source, detectors and digitizers) is compact. Additionally, the optical sensing device is amenable to multiplexed operation. Specifically in this Example, the optical sensing device was configured as a two-output system with a binding-specific sensor and a reference sensor, thereby enabling detection of an analyte along with an on-chip reference signal.

To fabricate the optical sensing devices, silicon oxynitride waveguide films were deposited in an Oxford Instruments Plasmalab 80 Plus capacitively coupled PECVD system at RF frequencies of 100 kHz. The refractive index of the waveguide film can be varied by gas composition and for this study the as-deposited waveguide films had a core index of n_{core}=1.8 and a thickness of t=330 nm for single mode operation. The waveguide films were deposited either on pyrex glass wafer substrates or on oxidized silicon wafer substrates (with a 10-μm oxide layer acting as lower cladding). Thermal nanoimprint lithography with commercial replica grating as the template followed by dry etching was used to integrate the grating pattern with the slab waveguide. The grating profile consisted of triangular grooves with a 1-μm pitch and a 80-nm profile depth as measured by atomic force microscopy (AFM).

Each optical sensing device was integrated in a fluidic cartridge consisting of a flow channel obtained in a silicon spacer and a lid with tubing connections. The optical sensing device was mounted to a rotary stage and the laser beam kept in a fixed position to select the light beam incident angle (typically 4 degrees for TM mode).

The light source utilized was a DFB laser module designed for the telecommunications market (Santur Corporation, model TL-2020-C). This laser has a small form factor (76×51×13 mm) while providing a wide tuning range. The laser provides a linearly polarized, fiber-coupled 20-mW power output with a wavelength range over 36 nm in the C-band (1528.77 to 1563.05 nm) and a channel spacing of 25 GHz (approximately 0.2 nm). The laser was computer-controlled via an RS-232 port. The laser beam was collimated by a spherical collimated lens package (Thorlabs, Inc., model F230FC-1550) and was unidimensionally expanded to a wide laser “strip” with a pair of cylindrical lenses with the focal length fat the f₁+f₂ distance (Thorlabs, Inc., model LJ1567L1-C, f₁=100 mm, and model LK1836L2-C f₂=−9.7 mm). The laser beam strip was incident on the grating at typically 4 degrees for TM mode and the wavelength was scanned. The wave-guided laser beam was outcoupled at the edge of the waveguide slab and collected by two 400-μm diameter optical fibers (Thorlabs, Inc.); the output fiber alignment had a large position tolerance (˜1 mm). The fibers were coupled to germanium photodiodes (Thorlabs, Inc., model SM05PD6A) with a 3-mm active area and no active circuitry. The photodiode current was digitized by a custom-made, multi-input 16-bit data acquisition board. LabVIEW® code was developed to integrate the data acquisition board photodetector reaction with the software interface controlling the laser wavelength sweep.

Wavelength spectra were acquired at the smallest wavelength step instrumentally available, 0.2 nm. The wavelength peak position was determined using a centroid method on linearly interpolated data. The optical sensing devices were exposed to aqueous solutions with different refractive indices to determine the volume refractive index sensitivity and limit of detection of the system. Adopting established conventions for refractive index sensing transducers, the sensitivity S is defined as wavelength shift per refractive index unit, and the detection limit is defined as R/S where R is the sensor resolution. The resolution is expressed in terms of standard deviation of noise of the sensor output. The sensitivity of the optical sensing devices was obtained by the slope of a linear fit of the data set as S=142 nm/RIU (refractive index unit). Typical values of the noise (standard deviation of baseline measurement) were 1-2 pm, corresponding to a detection limit of 1.0 10⁻⁵ RIU. This value compares well to the limits of detection of other optimized evanescent wave sensors. The simultaneous temporal response from the two sensors in response to different refractive-index samples revealed a coefficient of variation no larger than 15%. The surface sensing capability was demonstrated by detection of dilute (200 ng/ml) immunoglobulin protein samples binding to an activated sensor surface. The performance parameters of the optical sensing device are expected to be improved with further optimization of the device.

For purposes of the present disclosure, it will be understood that when a layer (or film, region, substrate, component, device, or the like) is referred to as being “on” or “over” another layer, that layer may be directly or actually on (or over) the other layer or, alternatively, intervening layers (e.g., buffer layers, transition layers, interlayers, sacrificial layers, etch-stop layers, masks, electrodes, interconnects, contacts, or the like) may also be present. A layer that is “directly on” another layer means that no intervening layer is present, unless otherwise indicated. It will also be understood that when a layer is referred to as being “on” (or “over”) another layer, that layer may cover the entire surface of the other layer or only a portion of the other layer. It will be further understood that terms such as “formed on” or “disposed on” are not intended to introduce any limitations relating to particular methods of material transport, deposition, fabrication, surface treatment, or physical, chemical, or ionic bonding or interaction. The term “interposed” is interpreted in a similar manner.

In general, terms such as “communicate” and “in . . . communication with” (for example, a first component “communicates with” or “is in communication with” a second component) are used herein to indicate a structural, functional, mechanical, electrical, signal, optical, magnetic, electromagnetic, ionic or fluidic relationship between two or more components or elements. As such, the fact that one component is said to communicate with a second component is not intended to exclude the possibility that additional components may be present between, and/or operatively associated or engaged with, the first and second components.

It will be understood that various aspects or details of the invention may be changed without departing from the scope of the invention. Furthermore, the foregoing description is for the purpose of illustration only, and not for the purpose of limitation—the invention being defined by the claims.

1. An optical sensing device for sensing analytes in a fluid sample, the optical sensing device comprising:
an optically transparent substrate;
a waveguide composed of a higher refractive-index material than the substrate and comprising a first surface disposed on the substrate, an opposing second surface, and an optical output edge between the first surface and the second surface, the first surface and the second surface parallel with a waveguide plane and the optical output edge substantially normal to the waveguide plane;
a diffraction grating formed on the waveguide; and
a plurality of sensors disposed on the diffraction grating and arranged in a 1×N array where N is an integer equal to or greater than 2, each sensor comprising a plurality of receptors immobilized on the diffraction grating, wherein at least one of the sensors is a binding-specific sensor comprising a plurality of binding-specific receptors, and
wherein the diffraction grating is configured for coupling a guided mode beam into the waveguide in response to an optical input beam incident on the sensors at a guided-mode resonance condition, the guided mode beam comprising N spatially distinct components that propagate along the waveguide plane from the respective sensors to the optical output edge.

2. The optical sensing device of claim 1, comprising an interlayer disposed on the substrate and composed of a material of lower refractive index than the waveguide and the substrate, wherein the first surface is disposed on the interlayer.

3. The optical sensing device of claim 1, wherein the diffraction grating has as configuration comprising at least one of:
the diffraction grating is unidiffractive;
the diffraction grating is multi-diffractive;
the diffraction grating is positioned at a distance from the optical output edge along the waveguide plane ranging from 1 to 40 mm;
the diffraction grating comprises a periodic structure having a pitch ranging from 250 nm to 2000 nm;
the diffraction grating comprises a plurality of grooves parallel with the optical output edge; or
a combination of two or more of the foregoing.

4.-6. (canceled)

7. The optical sensing device of claim 1, wherein the 1×N array is parallel with the optical output edge.

8. The optical sensing device of claim 1, wherein the plurality of sensors comprises at least one of:
binding-specific receptors comprising binding partners specific to the analytes;
first binding partners specific to second binding partners wherein the second binding partners are specific to the analytes;
a first binding-specific sensor and a second binding-specific sensor, the first binding-specific sensor comprising a plurality of binding-specific receptors of a first type, and the second binding-specific sensor comprising a plurality of binding-specific receptors of a second type different from the first type;
a reference sensor comprising a plurality of reference receptors; or
two or more of the foregoing.

9.-10. (canceled)

11. The optical sensing device of claim 1, comprising a fluidic structure disposed on the second surface and encapsulating the diffraction grating, the fluidic structure comprising an internal chamber communicating with the sensors, a fluid inlet communicating with the internal chamber, and a fluid outlet communicating with the internal chamber.

12. The optical sensing device of claim 11, wherein the plurality of sensors comprises at least one of:
a first binding-specific sensor and a second binding-specific sensor, wherein the internal chamber comprises a first flow channel communicating with the fluid inlet and the first binding-specific sensor, and a second flow channel communicating with the fluid inlet and the second binding-specific sensor;
a reference sensor communicating with the first flow channel or the second flow channel; or
both of the foregoing.

13. (canceled)

14. The optical sensing device of claim 11, wherein the fluid inlet comprises a plurality of separate inlet ports, the internal chamber comprises a plurality of flow channels communicating with the respective inlet ports, and each flow channel establishes a fluid flow path from the respective inlet port to one or more of the sensors.

15. An optical sensing apparatus for sensing analytes in a fluid sample, the optical sensing apparatus comprising:
an optical sensing device comprising an optically transparent substrate, a waveguide composed of a higher refractive-index material than the substrate and disposed on the substrate, a diffraction grating formed on the waveguide, and a plurality of sensors disposed on the diffraction grating, wherein the waveguide lies in a waveguide plane and comprises an optical output edge, the plurality of sensors is arranged in a 1×N array where N is an integer equal to or greater than 2, each sensor comprises a plurality of receptors immobilized on the diffraction grating, at least one of the sensors is a binding-specific sensor comprising a plurality of binding-specific receptors, and the diffraction grating is configured for coupling a guided mode beam into the waveguide in response to an optical input beam incident on the sensors at a guided-mode resonance condition, the guided mode beam comprising N spatially distinct guided mode components that propagate along the waveguide plane from the respective sensors to the optical output edge;
a wavelength-tunable light source configured for emitting the optical input beam at a wavelength that varies over a wavelength range at a controllable wavelength-varying rate, the wavelength-tunable light source positioned relative to the optical sensing device wherein the optical input beam propagates to the sensors at a fixed coupling angle; and
a plurality of optical detector units positioned for receiving respective N output beam components outcoupled from the optical output edge, the N output beam components corresponding to the N guided mode components.

16. The optical sensing apparatus of claim 15, comprising a beam expander interposed between the wavelength-tunable light source and the optical sensing device, and configured for expanding the optical input beam to a width sufficient for simultaneously irradiating all sensors of the 1×N array.

17. The optical sensing apparatus of claim 15, wherein the wavelength-tunable light source has a configuration comprising at least one of:
the wavelength-tunable light source is configured for emitting the optical input beam at one or more wavelengths in a wavelength range selected from the group consisting of the C-band, the L-band, and both the C-band and the L-band;
the wavelength-tunable light source is tunable over a wavelength range from 2 nm to 250 nm: or
both of the foregoing.

18. (canceled)

19. The optical sensing apparatus of claim 15, wherein the wavelength-tunable light source is positioned such that the coupling angle ranges from −20° to +20° relative to an axis normal to the waveguide plane.

20. The optical sensing apparatus of claim 15, wherein the plurality of optical detector units is arranged in a 1×N array parallel with the 1×N array of sensors.

21. (canceled)

22. A method for sensing analytes in a fluid sample, the method comprising:
bringing the fluid sample into contact with a plurality of sensors arranged in a 1×N array on a diffraction grating of a waveguide, where N is an integer equal to or greater than 2, each sensor comprising a plurality of receptors immobilized on the diffraction grating, wherein at least one of the sensors is a binding-specific sensor;
directing an optical input beam to the sensors at a fixed coupling angle;
while directing the optical input beam, scanning the optical input beam over a range of wavelengths, wherein at least one of the wavelengths satisfies a guided-mode resonance condition such that the diffraction grating couples a guided mode beam into the waveguide, the guided mode beam comprising N spatially distinct guided mode components that propagate along the waveguide plane from the respective sensors to an optical output edge of the waveguide;
receiving N output beam components outcoupled from the optical output edge at respective optical detector units, the N output beam components corresponding to the N guided mode components, to produce respective N signals proportional to respective intensities of the N output beam components at the scanned wavelengths; and
based on the received signals, determining whether a wavelength spectral shift in the guided mode beam has occurred, wherein the wavelength shift is indicative of a binding event occurring at the binding-specific sensor.

23. The method of claim 22, wherein bringing the fluid sample into contact with the plurality of sensors is done in accordance with an assay selected from the group consisting of a direct binding assay, a sandwich assay, a competitive assay, and an inhibition assay.

24. The method of claim 22, wherein the optical input beam is scanned over a range of infrared wavelengths.

25. The method of claim 22, wherein the optical input beam is scanned over a range of telecommunication wavelengths selected from the group consisting of wavelengths in the C-band, wavelengths in the L-band, and wavelengths in both the C-band and the L-band.

26. The method of claim 22, wherein at least one of the sensors is a reference sensor, at least one of the N output beam components is a reference beam component corresponding to the reference sensor, and at least one of the received signals is proportional to respective intensities of the reference beam component at the scanned wavelengths.

27. The method of claim 22, wherein at least one of the sensors is a reference sensor, and comprising: (a) determining whether a wavelength spectral shift has occurred in a guided mode beam component corresponding to the binding-specific sensor; (b) determining whether a wavelength spectral shift has occurred in a guided mode beam component corresponding to the reference sensor; and (c) based on the determinations of steps (a) and (b), determining whether a binding event has occurred at the binding-specific sensor that is indicative of the presence of analytes in the fluid sample.

28. The method of claim 22, comprising flowing a plurality of fluids to the waveguide through a plurality of respective inlet ports of a fluidic device encapsulating the sensors, and flowing the fluids from the waveguide through a common outlet port of the fluidic device, wherein flowing the fluids to and from the waveguide is done by operating a fluid moving device communicating with the common outlet port.

29. A method for detecting an infection caused by a pathogen to an organism, the method comprising:
bringing a physiological sample derived from the organism into contact with a first sensor and a second sensor disposed on a diffraction grating of a waveguide, the first sensor comprising a plurality of first receptors immobilized on the diffraction grating and the second sensor comprising a plurality of second receptors immobilized on the diffraction grating, wherein the first receptors are configured for binding specifically to a first binding partner selected from the group consisting of the pathogen and a biomarker indicative of the presence of the pathogen, and the second receptors are configured for binding specifically to a second binding partner indicative of an immunological response to the pathogen;
irradiating the first sensor and the second sensor with an optical input beam directed at a fixed coupling angle relative to the waveguide;
while irradiating, scanning the optical input beam over a range of wavelengths;
measuring intensities, as a function of the scanned wavelengths, of a first output beam component outcoupled from an edge of the waveguide, the first output beam generated in response to irradiation of the first sensor;
measuring intensities, as a function of the scanned wavelengths, of a second output beam component outcoupled from the edge, the second output beam generated in response to irradiation of the second sensor; and
based on the intensities measured, determining whether the first receptors have captured the first binding partner and whether the second receptors have captured the second binding partner.

30. The method of claim 29, wherein the first binding partner is the biomarker indicative of the presence of the pathogen, and the biomarker is selected from the group consisting of a nucleic acid of the pathogen, a coating protein, and a gene product of the pathogen.

31. The method of claim 29, wherein the second binding partner indicative of an immunological response to the pathogen is selected from the group consisting of an antibody against the pathogen, a cell surface marker, white blood cell marker, a chemokine, a cytokine, and a macrophage activation marker.
